SEQUENCE LISTING (8 sequences)

SEQ ID NO: 1 - DNA, 1672 bp, Valeriana officinalis
atgtctactgcattaaacagtgagcatgaaactgttcgtccattagcaagttttcaaccaagtacatggggcgatcttttcatctcttattctgaagatagccagcttaaggaagtatatggtaatgagcacgaatgtctgaaacaacaagtgaaaacaatgttgttggatgtgacaaattatagaatttccgagaaaatcgctttcataaatacgttggagagattaggggtatctcatgagtttgagaatgagattgaaggtctgcttcatcaaatgtttgatgctcattctaaattccaagatggtattcaacactttgatttgttcacattggggatttacttcaggattctcaggcaacatggctatagaatctattgtgatgttttcaacaagttgaaagatagcaacaatgaattcaagaaggaacttaaagaggacgcgatcggtttgctaagtttgtacgaagcaacacaagtaagagcacacgctgaagaaattttagacgaagccctcattttcacaaaggctcaacttgaatccatagccgcaacctccagcttaagcccatttgtcgagaagcaaattactcatgctttggtccaagctctccacaaaggaatcccaagagtcgaatcgcgccatttcatctctgtttatgaagaagatcctgacaaaaatgatttgttgttgaggttctcaaagattgattacaatattgtacaaatgcttcacaagcaagaattgtgccatatctcaaagtggtggagagattcggagctcgaaacaaaactaacttatgcgaggaatagagtggcggaatgctttttatggactctttgtgtgtaccacgaaccaaagtactctccggctcggcttctgttaggcaaactcataaatatcatatcttgcactgatgacacatatgatgcgtatggtacattagaggaagttcagatctttacagatgtcatacaaaggttggataggagttctatggagcagctgccggattacatgaaaatcctctacaaagctgtccttgatcttttcgacgaagtagaagttcagctatcgaaccaagaaactaataatacttatcgtatggcttatgccaaggaagagttaaaagctatcgccaagtgctacgaaaaggagcacatatggttcagaaaatgtcacgtgcccccattcgaagaatatctagagaatgcggtagtgtcaatcggtaatcgtttggccgtaactttttcttttctgggaatggatcaagtagcagctgttgaagcgttcgagtgggccaaaactgatcccaaaatggtaaaatcgtgcggtaaagtcttacgacttgttgacgacgtaatgagccacgaggaggaagatgtaagaggacacgtggcaacgggagtcgaatgctacatgaaagaacacggagtgagtagggaagaggccgtcgtggagttctacaagagggtcgagtacgcgtggaaggatgtgaacgaggaatttataacgccgaaccatctgcatatcgacctcctcaaccgcgttcttaaccttacaagaattgcagacgttgtttacaagtttgaagacggctacacgcaccccgagaagactctgaaacatcatatcatggcgttgttcgtcgaccccgtccccgtataga

SEQ ID NO: 2 - protein, 555 aa, Valeriana officinalis
MSTALNSEHETVRPLASFQPSTWGDLFISYSEDSQLKEVYGNEHECLKQQVKTMLLDVTNYRISEKIAFINTLERLGVSHEFENEIEGLLHQMFDAHSKFQDGIQHFDLFTLGIYFRILRQHGYRIYCDVFNKLKDSNNEFKKELKEDAIGLLSLYEATQVRAHAEEILDEALIFTKAQLESIAATSSLSPFVEKQITHALVQALHKGIPRVESRHFISVYEEDPDKNDLLLRFSKIDYNIVQMLHKQELCHISKWWRDSELETKLTYARNRVAECFLWTLCVYHEPKYSPARLLLGKLINIISCTDDTYDAYGTLEEVQIFTDVIQRLDRSSMEQLPDYMKILYKAVLDLFDEVEVQLSNQETNNTYRMAYAKEELKAIAKCYEKEHIWFRKCHVPPFEEYLENAVVSIGNRLAVTFSFLGMDQVAAVEAFEWAKTDPKMVKSCGKVLRLVDDVMSHEEEDVRGHVATGVECYMKEHGVSREEAVVEFYKRVEYAWKDVNEEFITPNHLHDLLNRVLNLTRIADVVYKFEDGYTHPEKTLKHHIMALFVDPVPV

SEQ ID NO: 3 - DNA, 1680 bp, Persicaria hydropiper
atgtctactgccgttaacgtcccatctgcggtccgccccgccgacaagcgtccgattgcgagctttcacccgagcccatggggcgactacttcctcaaatacgttccttgtgaccaggtgactcaagccaagatggaagatgaggtgaagaaagttgaagaggatgtaaagaaggagttgcggaagctggcgaaggctgtagggaagccattggagctgctcaacttcatcgatgtcgtcgaacgccttggggtgggataccgccttgagcaggagatcgaggaccttgttcaagctatattcgacaacgacaaatttggagtcgatgaattcgatctctatcatacttccctctggtttcgcctccttaggcaacatgggtttcacgtatcatgtgatgtgttcggaaaattcaagggcagaaacggaaggttcaaggactcgttggcgagtgatgtgaaggggatactcggcttgtacgaagcctcacatgttcgcacccatggcgatgacacgcttgatgaagcattggtgtttactacgactcatcttaaagccgtagtgaccaaccaaccaaaccatcccttggtgccacaagtgacccatgccctaatgcagccctaccacaagggcatgccaaggctcgagtctaggcacttcatcgcattctacgagaaagatccttaccacgacaaaaccttgttgaaatttggcaaattggacttcaacttggtgcaagcattgcacaagaaggagctcaaagatctcagcaggtggtggaaagatctagatatgcacgcgaagatgcctttcccgagcagagaccgagtgcccgaaggctacttttggacactagggcctttctatgaaccacaattcgctctttgtcgaaaatttttcttgcaagtgttcaaagtaacttccattgtcgatgatatctacgatgcctatggaactatcgatgagctcaccgctttcactaaagctgctgagagatgggatcgtagttgccttgatgaacttccggaatacatgaaagtgagctacgcgtctctcattgataccttcgaggaatttgaacgcgacttggctccccaaggaagatcttggagcgtcaagtacgcaagagaggaaatgatacagatgtgtagagtttactaccaagaagcgaaatggtgccatgagaaatactcgcccacctgcgacgagtacttggagaaagcatccatagtgagtttcggctacaacttgggaacagtagtgtgcttcctcgggatgggagacgtcgctacaaaggaggcattcgaatgggctcgcggaaacccaaaggtcgtaagagccgcaggcataatcggaaggctcatggacgacataggcagccatcattttgagcaaggtagagaccatgttccatccgccgtggagtgctacataaggcagcacggtgtcgacgaagtaaccgcccaaagagagttgggaaagcgggtggaaagtagctggaaggacatcaatgagatgatgttgaagccttatatgatgccgaagcctcttctaactcgcatccttaacgagtgtcgcattgtggatgtgatctacaagggagaagatagctacaccttctccaacaccaccatgaagaaaaacatttctcacattctcaccgatcccattccgatttag

SEQ ID NO: 4 - protein, 558 aa, P. hydropiper
MSTAVNVPSAVRPADKRPIASFHPSPWGDYFLKYVPCDQVTQAKMEDEVKKVEEDVKKELRKLAKAVGKPLELLNFIDVVERLGVGYRLEQEIEDLVQAIFDNDKFGVDEFDLYHTSLWFRLLRQHGFHVSCDVFGKFKGRNGRFKDSLASDVKGILGLYEASHVRTHGDDTLDEALVFTTTHLKAVVTNQPNHPLVPQVTHALMQPYHKGMPRLESRHFIAFYEKDPYHDKTLLKFGKLDFNLVQALHKKELKDLSRWWKDLDMHAKMPFPSRDRVPEGFWTLGPFYEPQFALCRKFFLQVFKVTSIVDDIYDAYGTIDELTAFTKAAERWDRSCLDELPEYMKVSYASLIDTFEEFERDLAPQGRSWSVKYAREEMIQMCRVYYQEAKWCHEKYSPTCDEYLEKASIVSFGYNLGTVVCFLGMGDVATKEAFEWARGNPKVVRAAGIIGRLMDDIGSHHFEQGRDHVPSAVECYIRQHGVDEVTAQRELGKRVESSWKDINEMMLKPYMMPKPLLTRILNECRIVDVIYKGEDSYTFSNTTMKKNISHILTDPIPI

SEQ ID NO: 5 - protein, 437 aa, Arabidopsis thaliana
MDPTESLPEEDEEIVKSVIDGVIPSYSLESRLGDCKRAASIRREALQRVTGRSIEGLPLDGFDYESILGQCCEMPVGYIQIPVGIAGPLLLDGYEYSVPMATTEGCLVASTNRGCKAMFISGGATSTVLKDGMTRAPVVRFASARRASELKFFLENPENFDTLAVVFNRSSRFARLQSVKCTIAGKNAYVRFCCSTGDAMGMNMVSKGVQNVLEYLTDDFPDMDVIGISGNFCSDKKPAAVNWIEGRGKSVVCEAVIRGEIVNKVLKTSVAALVELNMLKNLAGSAVAGSLGGFNAHASNIVSAVFIATGQDPAQNVESSQCITMMEAINDGKDIHISVTMPSIEVGTVGGGTQLASQSACLNLLGVKGASTESPGMNARRLATIVAGAVLAGELSLMSAIAAGQLVRSHMKYNRSSRDISGATTTTTTTTAAADLQ

SEQ ID NO: 6 - protein, 342 aa, Arabidopsis thaliana
MADLKSTFLDVYSVLKSDLLQDPSFEFTHESRQWLERMLDYNVRGGKLNRGLSVVDSYKLLKQGQDLTEKETFLSCALGWCIEWLQAYFLVLDDIMDNSVTRRGQPCWFRKPKVGMIAINDGILLRNHIHRILKKHFREMPYYVDLVDLFNEVEFQTACGQMIDLITTFDGEKDLSKYSLQIHRRIVEYKTAYYSFYLPVACALLMAGENLENHTDVKTVLVDMGIYFQVQDDYLDCFADPETLGKIGTDIEDFKCSWLVVKALERCSEEQTKILYENYGKAEPSNVAKVKALYKELDLEGAFMEYEKESYEKLTKLIEAHQSKAIQAVLKSFLAKIYKRQK

SEQ ID NO: 7 - DNA, 72 bp, Saccharomyces cerevisiae
atgttgtcactacgtcaatctataagatttttcaagccagccacaagaactttgtgtagctctcgttatctg

SEQ ID NO: 8 - DNA, 289 bp, Ch. morifolium
atggcctcgatctcttcctccgctgtcgcaaccgtcaacaggaccacctctgctcaagctagcatggtggctccattcaccgggcttaagtccaacgtcgctttcccagtcaccaagaagtctaacgacttctcatccctccccagcaacggtggaagagtgcaatgcatgaaggtacaatatataacttaaataataacgtgaacacttattataatgcagtagatataatgactaacattttataaaatatatatataggtgtggccaccattgggtttgaagaagt

(19) United States
(12) Patent Application Publication
(10) Pub. No.: US 2015/0059018 A1
(43) Pub. Date: Feb. 26, 2015

(54) METHODS AND COMPOSITIONS FOR PRODUCING DRIMENOL

(71) Applicant: Keygene N.V., Wageningen (NL)

(72) Inventors: Hendrik Jan Bouwmeester, Wageningen (NL); Maurice Gerard Leon Henquet, Wageningen (NL); Maarten Anthonie Jongsma, Wageningen (NL)

(21) Appl. No.: 14/352,672

(22) PCT Filed: Oct. 19, 2012
(86) PCT No.: PCT/NL2012/050730

§ 371 Date: Apr. 17, 2014

Related U.S. Application Data
(60) Provisional application No. 61/548,934, filed on Oct. 19, 2011.

(30) Foreign Application Priority Data
Oct. 20, 2011 (NL) 2007626

(51) Int. Cl. C12P 7/02; C12N 9/16

(52) U.S. Cl. CPC C12P 7/02; C12N 9/16
USPC 800/278; 536/23.2; 435/196; 435/320.1; 435/325; 435/252.3; 435/254.11; 435/419; 800/298; 435/155; 800/317.4; 800/317.1; 800/317; 800/305; 800/322; 800/306; 800/307; 800/309; 800/310; 800/314; 800/312; 800/313; 800/317.2; 800/320.1; 800/320; 800/320.3; 800/320.2; 800/308

(57) ABSTRACT

The present invention relates to nucleic acids sequences derived from Valeriana officinalis and/or Persicaria hydropiper and encoding drimenol synthase polypeptides. The present invention also provides the amino acid sequences of the polypeptides. The invention further provides host cells or organisms genetically modified to harbour the polynucleotides of the invention. A method to produce drimenol and/or a drimenol derivative by contacting farnesyl diphosphate with a polypeptide having a drimenol synthase activity is also part of this invention.

### FIELD OF THE INVENTION

The present invention relates to nucleic acid molecules isolated from Valeriana officinalis and Persicaria hydropiper and encoding drimenol synthase proteins, expression vectors comprising the nucleic acid molecules, chimeric genes comprising the nucleic acid molecules, host cells altered to harbour the nucleic acid molecules and drimenol synthase proteins themselves. The invention herein further provides methods for producing drimenol, or drimenol derivatives in the cells or organisms harbouring such nucleic acid molecules or by contacting the precursors with the polypeptides isolated from such cells or organisms. Transgenic organisms comprising the nucleic acid molecules of the invention are also provided. The present invention especially relates to transgenic plants with enhanced resistance to insects.

### BACKGROUND OF THE INVENTION

Drimenol was reported to have plant growth regulatory activity comparable to that of heteroauxin indole-3-acetic acid. Importantly, drimenol has been often used as a starting compound in the organic synthesis of diverse biologically active drimanes and nordrimanes that have limited availability from natural sources. Of particular interest is polygodial, a drimane dialdehyde, chemically and naturally synthesized from drimenol. Polygodial was reported to have antifeedant, antibacterial, antifungal, cytotoxic, allergenic, piscicidal, molluscicidal, analgetic and plant growth regulatory activities. Drimenol has been used as a starting compound for synthesis of other drimanic sesquiterpenes with active biological properties such as warburganal, a drimanic dialdehyde, which is similar to polygodial in the variety of useful biological properties, and (−)-cinnamodial, which possess antimicrobial, antifeedant, piscicidal, anthelmintic activities (Jansen and de Groot 1991 Nat Prod Rep 8: 309; Jansen and de Groot 2004 Nat Prod Rep 21: 449). Drimenol has been also used for production of nordrimanic compounds such as fragrant hydroxyl ketones.

Metabolic engineering of terpenes in plants by overexpressing enzymes catalyzing steps in the terpene biosynthesis pathway was shown to be successful to generate substantial levels of terpenes.

Terpenes are synthesized from the common precursor isopentenyl pyrophosphate (IPP) and its isomer dimethylallyl diphosphate (DMAPP) through two distinct biosynthesis pathways. Generally, sesquiterpenes are synthesized from the relevant precursors through the mevalonate pathway in the cytosol, and monoterpenes and diterpenes are produced through the DXP pathway in plastids. Exchange of precursors between plastids and cytosol is also observed.

In both pathways, the IPP is further isomerized to DMAPP by the IPP isomerase with subsequent formation of the higher molecular weight acyclic polyprenyl pyrophosphate precursors by prenyl transferases to form the acyclic pyrophosphate terpene precursors. For example, these reactions produce ten-, fifteen-, and twenty-carbon precursors geranyl-pyrophosphate (GPP), farnesyl-pyrophosphate (FPP), geranylgeranyl-pyrophosphate (GGPP), respectively. Terpene synthases are the enzymes catalyzing the cyclisation of the acyclic precursors in the multi-step reactions producing the carbon skeleton of terpene, monoterpene or sesquiterpene compounds. For example, the initial step of the catalyzed cyclisation may be the ionization of the diphosphate group to form an allylic cation. The substrate then undergoes isomerizations and rearrangements which can be controlled by the active site of an enzyme. The product, for example, may be an acyclic, mono-, di or tricyclic terpene.

It is known in the art that GPP and neryl diphospate (NPP), the cis-isomer of GPP, are the substrates for monoterpene biosynthesis, and that FPP and GGPP are the respective substrates for sesquiterpene synthases and diterpene synthases (Chen et al., 2011 Plant J 66:212-229; Schilmiller et al., 2009 Proc Natl Acad Sci 106:10865-10870; Tholl 2006 Curr Opin Plant Biol 9:297-304; Wang and Ohnuma, 2000 Biochim Biophys Acta 1529:33-48). Some terpene synthases produce a single product, but many produce multiple products from the same precursor, or can produce multiple compounds depending on the precursor supplied (Van Schie et al., 2007 Plant Mol Biol 64:251-263).

Induced terpene biosynthesis was observed to correlate with induced expression of terpene synthases (Navia-Gine et al., 2009 Plant Phys Biochem 47: 416-425; Herde et al., 2008 Plant Cell 20: 1152-1168).

Several terpene synthases have been identified (WO2010/064897 and WO2009/044336). Previously, a partially purified protein from Persicaria hydropiper was identified as a drimenol cyclase (Banthorpe et al. 1992 Phytochemistry 31: 3391). However, this reference did not provide the amino acid sequence of the protein, nucleotide sequence of a gene encoding it, or any methods for producing drimenol or its derivatives. WO2004031376 provides plant sesquiterpene synthase and methods for making and using these enzymes for the production of various oxygenated and aliphatic sesquiterpenes including valencene, bicyclo-germacrene, cubebol, and delta-cadine. Jones et al reports three sesquiterpene synthases that were isolated from different species of the genus Santalum (sandalwood), and which were cloned using primers of previously amplified terpene synthase from Santalum album (Jones et al. 2011 Journal of Biological Chemistry, Vol 286 pp. 17445-17454). However, neither of these references provide any nucleotide sequence encoding drimenol synthase or any method for producing drimenol synthase, and/or drimenol, and/or drimenol derivatives.

Like other drimanic compounds, drimenol may be isolated from natural sources. However, this approach is of little utility, due to the low content of drimenol and the difficulty of its isolation and purification. Therefore, much attention has been focused on alternative ways to increase production of drimenol.

### SUMMARY OF THE INVENTION

The present invention provides the drimenol synthase genes cloned from V. officinalis and P. hydropiper and drimenol synthase proteins that can be used for in vitro or in vivo production of drimenol or drimenol derivatives.

An aspect of the invention herein is an isolated polypeptide having drimenol synthase activity and comprising the amino acid sequence of SEQ ID NO:2 or an amino acid sequence that is at least 70%, 75%, 80%, 85%, 90%, 92%, 95%, 96%, 97%, 98% or 99% identical to SEQ ID NO:2. An isolated nucleic acid encoding such polypeptide, or variants, or fragments thereof is also provided and, for example, includes the nucleotide sequence of SEQ ID:1. In certain embodiments, the isolated nucleic acid is derived from Valeriana officinalis.

An alternative embodiment of the invention herein provides an isolated polypeptide having drimenol synthase activity and comprising the amino acid sequence of SEQ ID NO: 4, or an amino acid sequence that is at least 70%, 75%, 80%, 85%, 90%, 92%, 95%, 96%, 97%, 98% or 99% identical to the amino acid sequence of SEQ ID NO:4. An isolated nucleic acid encoding the polypeptide having the amino acid sequence of SEQ ID NO:4, or variants, or fragments thereof is also within the scope of the invention. For example, an isolated nucleic acid includes the nucleotide sequence of SEQ ID:3, and, in certain embodiments, is derived from Persicaria hydropiper. The invention also pertains to a chimeric gene comprising a nucleic acid sequence of the invention.

An embodiment of the invention provides an expression vector that includes the nucleotide sequence of SEQ ID NO:1 and/or SEQ ID NO:3, or a chimeric gene comprising the nucleotide sequence of SEQ ID NO:1 and/or SEQ ID NO:3 In general, the expression vector and/or chimeric gene include the nucleotide sequence operably linked to at least one regulatory sequence which controls transcription, translation initiation or termination. Alternatively, the expression vector and/or the chimeric gene include the nucleotide sequence operably linked to at least one promoter with constitutive activity, or at least one inducible promoter, or at least one insect-inducible promoter, which controls transcription.

In certain other embodiments, the nucleic acid sequence of the expression vector and/or chimeric gene further includes a targeting sequence. For example, the targeting sequence is a transit peptide that targets the polypeptide product of the nucleic acid sequence to a plastid of the plant cell. For example, the plastid is a chloroplast. Alternatively, the targeting sequence is a transit peptide that targets the polypeptide product of the nucleic acid sequence to a mitochondrion of a plant cell.

Another aspect of the invention is a method for producing drimenol, or at least one drimenol derivative, such method including the steps of: (a) contacting a farnesyl-pyrophosphate (FPP) precursor with a polypeptide having drimenol synthase activity and having the amino acid sequence of SEQ ID NO:2 or SEQ ID NO:4, or an amino acid sequence at least 70%, 75%, 80%, 85%, 90%, 92%, 95%, 96%, 97%, 98% or 99% identical to SEQ ID NO:2, or SEQ ID NO:4; (b) isolating drimenol; and (c) optionally, contacting drimenol produced in steps (a), (b) with at least one enzyme converting drimenol into the at least one drimenol derivative.

An embodiment of the method further provides a step prior to step (a) transforming or transfecting a host cell capable of producing the FPP precursor with a nucleic acid sequence encoding the polypeptide of the invention.

In a preferred embodiment of the method, step (a) is carried out by culturing the cell under conditions permitting production of drimenol and/or the drimenol derivative. In general, the cell is selected from the group of: plant cells, bacterial cells, and fungal cells.

An alternative embodiment of the method further includes hydroxylation and/or oxidation of drimenol to produce at least one drimenol derivative having fungicidal, insecticidal, antifeedant, fragrance and/or food taste modifying properties. For example, the drimenol derivative is selected, but not limited to, from the group comprising driman-8-ol, driman-8,11-diol, drim-8-en-7-one, forskolin, cinnamodial, (+)-albicanol, (−)-uvidin, (+)-isopolygonal, (−)-polygodial, (−)-ugandensidial, (−)-warburganal, ambergris, drimenal, drimenoic acid, isodrimenin, cinnamolide, confertolin, confertifolin, drimendiol, and polygodial acid.

A transgenic organism that includes any of the isolated nucleic acid sequences of the invention herein is also provided by embodiments of the invention. In general, the organism comprises a plant, a micro-organism or a fungus.

Yet another aspect of the invention provides a method for producing at least one polypeptide having drimenol synthase activity including the steps of: a) transforming a host cell or a non-human organism with any of the nucleic acid sequences or the expression vectors or chimeric genes of the invention herein; and b) culturing the cell or the organism under conditions permitting production of a polypeptide of the invention.

Another embodiment provides a method for producing a transgenic plant capable of producing drimenol and/or increased levels of drimenol compared to a non-transgenic plant of a similar genetic background, the method comprising the steps of: a) transforming a plant or a plant cell with a nucleic acid encoding a polypeptide having drimenol synthase activity and comprising the amino acid sequence of SEQ ID NO:2 or SEQ ID NO:4, or an amino acid sequence that is at least 70%, 75%, 80%, 85%, 90%, 92%, 95%, 96%, 97%, 98% or 99% identical to the amino acid sequence of SEQ ID NO:2, or SEQ ID NO: 4, or a chimeric gene comprising a nucleic acid encoding a polypeptide having drimenol synthase activity and comprising the amino acid sequence of SEQ ID NO:2 or SEQ ID NO:4 or an amino acid sequence that is at least 70%, 75%, 80%, 85%, 90%, 92%, 95%, 96%, 97%, 98% or 99% identical to the amino acid sequence of SEQ ID NO:2 or SEQ ID NO:4, operably linked to a promoter, and b) regenerating a plant. For example, the promoter may be a 35S promoter. In another embodiment, the promoter is insect-inducible.

In an embodiment, said transgenic plant has enhanced insect resistance compared to a non-transgenic plant of a similar genetic background.

An alternative embodiment of the method further includes screening the transgenic plant, or a plant derived therefrom by selfing or crossing, for production of drimenol and identifying the plant producing drimenol. For example, such transgenic plant is a crop plant.

### DETAILED DESCRIPTION OF THE INVENTION

### General Definitions

The term “polypeptide” means an amino acid sequence of consecutively polymerized amino acid residues, for instance, at least 15 residues, at least 30 residues, at least 50 residues. In some embodiments of the invention, a polypeptide comprises an amino acid sequence that is an enzyme, or a fragment, or a variant thereof.

The term “isolated” polypeptide refers to an amino acid sequence that is removed from its natural environment by any method or combination of methods known in the art and includes recombinant, biochemical and synthetic methods.

The term “protein” refers to an amino acid sequence of any length wherein amino acids are linked by covalent peptide bonds, and includes oligopeptide, peptide, polypeptide and full length protein whether naturally occurring or synthetic.

The terms “drimenol synthase” or “drimenol synthase protein” refer to an enzyme that is capable of converting farnesyl diphosphate (FPP) to drimenol.

The terms “biological function,” “function,” “biological activity” or “activity” refer to the ability of the drimenol synthase of the present invention to catalyze the formation of drimenol from FPP.

The terms “nucleic acid sequence,” “nucleic acid,” and “polynucleotide” are used interchangeably meaning a sequence of nucleotides. A nucleic acid sequence may be a single-stranded or double-stranded deoxyribonucleotide, or ribonucleotide of any length, and include coding and non-coding sequences of a gene, exons, introns, sense and anti-sense complimentary sequences, genomic DNA, cDNA, miRNA, siRNA, mRNA, rRNA, tRNA, recombinant nucleic acid sequences, isolated and purified naturally occurring DNA and/or RNA sequences, synthetic DNA and RNA sequences, fragments, primers and nucleic acid probes. The skilled artisan is aware that the nucleic acid sequences of RNA are identical to the DNA sequences with the difference of thymine (T) being replaced by uracil (U).

An “isolated nucleic acid” or “isolated nucleic acid sequence” is defined as a nucleic acid or nucleic acid sequence that is in an environment different from that in which the nucleic acid or nucleic acid sequence naturally occurs, i.e. substantially separated from other cellular components, like ribosomes, polymerases and many other genome sequences which naturally accompany such nucleic acid in a cell in which it naturally occurs. The term “naturally-occurring” as used herein as applied to a nucleic acid refers to a nucleic acid that is found in a cell in nature. For example, a nucleic acid sequence that is present in an organism, for instance in the cells of an organism, that can be isolated from a source in nature and which has not been intentionally modified by a human in the laboratory is naturally occurring.

“Recombinant nucleic acid sequence” are nucleic acid sequences that result from the use of laboratory methods (molecular cloning) to bring together genetic material from more than on source, creating a nucleic acid sequence that does not occur naturally and would not be otherwise found in biological organisms.

“Recombinant DNA technology” refers to molecular biology procedures to prepare a recombinant nucleic acid sequence as described, for instance, in Laboratory Manuals edited by Weigel and Glazebrook, 2002 Cold Spring Harbor Lab Press; and Sambrook et al., 1989 Cold Spring Harbor, N.Y.: Cold Spring Harbor Laboratory Press.

The term “gene” means a DNA sequence comprising a region, which is transcribed into a RNA molecule, e.g., an mRNA in a cell, operably linked to suitable regulatory regions, e.g., a promoter. A gene may thus comprise several operably linked sequences, such as a promoter, a 5′ leader sequence comprising, e.g., sequences involved in translation initiation, a coding region of cDNA or genomic DNA, introns, exons, and/or a 3′non-translated sequence comprising, e.g., transcription termination sites.

A “chimeric gene” refers to any gene, which is not normally found in nature in a species, in particular, a gene in which one or more parts of the nucleic acid sequence are present that are not associated with each other in nature. For example the promoter is not associated in nature with part or all of the transcribed region or with another regulatory region. The term “chimeric gene” is understood to include expression constructs in which a promoter or transcription regulatory sequence is operably linked to one or more coding sequences or to an antisense, i.e., reverse complement of the sense strand, or inverted repeat sequence (sense and antisense, whereby the RNA transcript forms double stranded RNA upon transcription).

A “3′ UTR” or “3′ non-translated sequence” (also referred to as “3′ untranslated region,” or “3′end”) refers to the nucleic acid sequence found downstream of the coding sequence of a gene, which comprises for example a transcription termination site and (in most, but not all eukaryotic mRNAs) a polyadenylation signal such as AAUAAA or variants thereof. After termination of transcription, the mRNA transcript may be cleaved downstream of the polyadenylation signal and a poly(A) tail may be added, which is involved in the transport of the mRNA to the site of translation, e.g., cytoplasm.

“Homology” refers to a sequence similarity, or identity between a polypeptide or a fragment thereof and a references sequence. A homology of polypeptide sequences are determined based on the number of amino acid sequences in the positions shared by the polypeptides. Homologous sequences encompass amino acid sequences of polypeptide of the present invention modified by chemical or enzymatic means known in the art. See Ausubel et al. (eds) 2000 Current Protocols Mol Biol, Willey & Sons, New York.

“Expression of a gene” involves transcription of the gene and translation of the mRNA into a protein. Overexpression refers to the production of the gene product as measured by levels of mRNA, polypeptide and/or enzyme activity in transgenic cells or organisms that exceeds levels of production in non-transformed cells or organisms of a similar genetic background.

“Expression vector” as used herein means a nucleic acid molecule engineered using molecular biology methods and recombinant DNA technology for delivery of foreign or exogenous DNA into a host cell. The expression vector typically includes sequences required for proper transcription of the nucleotide sequence. The coding region usually codes for a protein of interest but may also code for an RNA, e.g., an antisense RNA, siRNA and the like.

“Regulatory sequence” refers to a nucleic acid sequence that determines expression level of the nucleic acid sequences of the invention and is capable of regulating the rate of transcription of the nucleic acid sequence operably linked to the regulatory sequence. Regulatory sequences comprise promoters, enhancers, transcription factors, promoter elements and the like.

“Promoter” refers to a nucleic acid sequence that controls the expression of a coding sequence by providing a binding site for RNA polymerase and other factors required for proper transcription including without limitation transcription factor binding sites, repressor and activator protein binding sites. The meaning of the term promoter also include the term “promoter regulatory sequence”. Promoter regulatory sequences may include upstream and downstream elements that may influences transcription, RNA processing or stability of the associated coding nucleic acid sequence. Promoters include naturally-derived and synthetic sequences. The coding nucleic acid sequences is usually located downstream of the promoter with respect to the direction of the transcription starting at the transcription initiation site.

The term “constitutive promoter” refers to an unregulated promoter that allows for continual transcription of the nucleic acid sequence it is operably linked to.

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked means that the DNA sequences being linked are typically contiguous. The nucleotide sequence associated with the promoter sequence may be of homologous or heterologous origin with respect to the plant to be transformed. The sequence may be also entirely or partially synthetic. Regardless of the origin, the nucleic acid sequence associated with the promoter sequence will be expressed or silenced in accordance with promoter properties to which it is linked after binding to the polypeptide of the invention. The associated nucleic acid may code for a protein that is desired to be expressed or suppressed throughout the plant at all times or, alternatively, in specific cells and tissues. Such nucleotide sequences preferably encode proteins conferring desirable phenotypic traits to the host cells or organism altered or transformed therewith. More preferably, the associated nucleotide sequence leads to the production of drimenol in the plant. Preferably, the nucleotide sequence encodes drimenol synthase.

“Target peptide” refers to an amino acid sequence which targets a protein, or polypeptide to intracellular organelles, i.e., mitochondria, or plastids, or to the extracellular space or apoplast (secretion signal peptide). A nucleic acid sequence encoding a target peptide may be fused to the nucleic acid sequence encoding the amino terminal end, e.g., N-terminal end, of the protein or polypeptide, or may be used to replace a native targeting polypeptide.

The term “percentage of identity” refers to a statistical measure of the degree of relatedness of two protein sequences. The percentage of sequence identity between two sequences is determined using computer programs that are based on standard alignment algorithms. Sequences are substantially identical when they share at least a certain minimal percentage of sequence identity as identified by standard computer programs. Computer programs that are preferred within the scope of the present invention include without limitation the CGC program package (Devereux et al., 1984 Nucleic Acid Research 12:387), BestFit, BLASTP, BLASTN, and FASTA (Altshul et al., 1990 J Mol Biol 215: 403), the algorithm of Meyers et al., 1988 Comput Appl Biosci 4:11, or the algorithm of Needleman et al., 1970 J Mol Biol 48:443. Preferably, the sequence identity refers to the sequence identity over the entire length of the sequence.

The term “primer” refers to a short nucleic acid sequence that is hybridized to a template nucleic acid sequence and is used for polymerization of a nucleic acid sequence complementary to the template.

As used herein, the term “host cell” or “transformed cell” refers to a cell (or organism) altered to harbor at least one nucleic acid molecule, for instance, a recombinant gene encoding a desired protein or nucleic acid sequence which upon transcription yields a drimenol synthase protein useful to produce drimenol. The host cell is preferably a bacterial cell, a fungal cell or a plant cell. The host cell may contain a recombinant gene according to the present invention which has been integrated into the nuclear or organelle genomes of the host cell. Alternatively, the host may contain the recombinant gene extra-chromosomally.

The term “selectable marker” refers to any gene which upon expression may be used to select a cell or cells that include the selectable marker. Examples of selectable markers are described below. The skilled artisan will know that different antibiotic or herbicide selectable markers are applicable to different target species. Selectable markers that are routinely used in plant transformation include the npt II gene conferring resistance to kanamycin, paromymycin, geneticin, and related antibiotics the bacterial aad. A gene encoding aminoglycoside 3′-adenyltransferase conferring resistance to antibiotics streptomycin or spectinomycin, the hph gene conferring resistance to hygromycin. Other markers that can be used include a mutant EPSP gene conferring resistance to glyphosate, a mutant acetolactate synthase (ALS) gene conferring resistance to imidazoline or sulphonylurea herbicides, a phospinothricin acetyltransferase gene which confers resistance to herbicide phosphinothricin. Selection markers resulting in positive selection such as phosphomannose isomerase gene may also used (see WO 93/05163).

The term “drimenol” is used to denote any drimenol molecule having a formula C₁₅H₂₆O including (−)-drimenol (CAS: 468-68-8) and is intended to also include drimenol and drimenol derivatives, for example as mentioned herein, including compounds derived from drimenol that have undergone one or more steps of hydroxylation, oxidation, acetylation, isomerisation, dimethylation and the like. As used herein a “derivative” refers to any compound obtained from drimenol and containing essential elements of the parent substance, and includes without limitation driman-8-ol, driman-8,11-diol, drim-8-en-7-one, forskolin, cinnamodial, (+)-albicanol, (−)-uvidin, (+)-isopolygonal, (−)-polygodial, (−)-ugandensidial, (−)-warburganal, ambergris, drimenal, drimenoic acid, isodrimenin, cinnamolide, confertolin, confertifolin, drimendiol, and polygodial acid.

Similarly, the term “polygodial” refers to any type of polygodial molecule of a formula C₁₅H₂₂O₂. In nature, polygodial is made in at least two steps from farnesyl diphosphate, first by the enzyme drimenol synthase, and subsequently by a P450 enzyme to first introduce a hydroxylation and subsequently oxidation into two aldehydes on the drimenol backbone.

The term “organism” refers to any non-human multicellular or unicellular organisms such as a plant, or a microorganism. Preferably, a micro-organism is a bacterium, a yeast, an algae or a fungus.

The term “plant” is used interchangeably to include plant cells including plant protoplasts, plant tissues, plant cell tissue cultures giving rise to regenerated plants, or parts of plants, or plant organs such as roots, stems, leaves, flowers, pollen, ovules, embryos, fruits and the like. Any plant can be used to carry out the methods of the invention. Preferably, the plant is selected from the family Solanaceae, Valerianaceae, Malvaceae, Asteraceae, Brassicaceae, Polygonaceae, Poaceae (formerly Gramineae) or Fabaceae.

The term “crop species” refers to plants cultivated for purposes of obtaining food, feed or plant derived products including carbohydrates, oils and medicinal ingredients.

As used herein, a “genetic background” refers to the genotypic base of a breeding line or population of organisms.

The terms “plant insects” or “plant pests” refer to insect species that infest and damage host crop and ornamental plants. An “infestation” refers to presence of a large number of pest organisms in a field or greenhouse, on the surface of a host plant or on anything that might contact a host plant, or in the soil. Insect pests include sap-sucking insect pests, such as psyllids, whiteflies, aphids, mealybugs, plant hoppers and scale insects and share a common property, namely the utilization of plant sap as their food source. Insect pests also include thrips, cicada, mites and leaf hoppers.

The term “insect pests” also refers herein to insects of the order Diptera including but not limiting to blood sucking or biting insects attacking animals, especially mammals. Sap sucking insects and blood sucking ticks are also included. Such insects or arachnids may act as vectors of human and/or mammalian diseases such as malaria.

The term “whitefly” or “whiteflies” refers to species of the genus Bemisia, especially B. tabaci, species of the genus Trialeurodes, especially the greenhouse whitefly T. vaporariorum and the banded winged whitefly T. abutinolea. All biotypes of B. tabaci such as biotype Q and B, are also included as well as any developmental stage, such as eggs, larvae, pupae and adults.

As used herein, the term “aphids” refers to plant insect pests belonging to the family Aphididae, including but not limited to Aphis gossypii, A. fabae, A. glycines, A. nerii, A. nasturtii, Myzus persicae, M. cerasi, M. ornatus, Nasonovia especially N. ribisnigri, Macrosiphum, and Brevicoryne.

The term “antifeedant” refers to a compound that inhibits feeding but does not kill the “insect pest” directly, although it may lead to the insect's death by starvation. The terms “feeding deterrent” or “gustatory repellent” are synonymous with antifeedant. However, the term “antifeedant” is not synonymous with the term “olfactory repellent”, which is usually a volatile compound which repels the insect before it starts to feed on the plant. For example, warburganal produced by Warburgia stuhlmannii, is a specific antifeedant against larvae of the African army worm but it may not have any repellent effect against other insects.

The term “insect vectors” refers to insects that are capable of carrying and transmitting viruses to plants. In the context of plant disease vectors, insect vectors are insects which attack plants and can potentially transmit diseases to plants, such as sap sucking insects whiteflies and aphids, which are able to transmit diseases to plants. Preferably, the modified plants of the invention develop enhanced resistance to one or more pest insects.

### Polypeptides of the Invention

It is an object of the present invention to provide new polynucleotide sequences encoding drimenol synthase proteins, methods for in vitro and in vivo synthesis of drimenol and/or a drimenol derivative using proteins of the invention and methods of genetic modification of organisms, especially plants, to alter levels of drimenol synthase activity, and/or to alter levels of drimenol and/or drimenol derivatives.

An embodiment of the invention provides a drimenol synthase, drimenol synthase homologous polypeptides, and variants thereof. The polypeptides of the invention herein catalyze production of drimenol from a FPP precursor.

A related embodiment of the invention provides an isolated or recombinant polypeptide which has the amino acid sequence set forth in SEQ ID NO:2, or SEQ ID NO:4, or fragments, or variants or derivatives thereof. Preferably, the variants will possess at least 55% identity to the polypeptides of SEQ ID NO:2 or SEQ ID NO:4 over the entire length of the respected polypeptide, and preferably the variants will possess at least 70%, 75%, 80%, 85%, 90%, 92%, 95%, 96%, 97%, 98% or 99% identity over the entire length of the polypeptides of the invention.

Fragments of a polypeptide having the amino acid sequence of SEQ ID NO:2 or SEQ ID NO:4 or variants thereof are subsequences of the polypeptide of the invention that retain drimenol synthase activity and capacity to catalyze the formation of drimenol from FPP. The term “fragment” may refer to a recombinant polypeptide and/or an aggregate polypeptide such as a dimer or multimer. Fragments of the drimenol synthase protein according to this invention may comprise fragments of 100, 150, 200, 300, 400, 500 contiguous amino acids or more. Preferably these fragments retain drimenol synthase activity in non-human organisms and are capable of producing drimenol from FPP in a host cell or organism.

Also included are amino acid sequences which share homology with the polypeptides having the amino acid sequence of SEQ ID NO:2 or SEQ ID NO: 4. Homologous sequences are sequences that share substantial sequence identity or similarity to the amino acid sequence of SEQ ID NO: 2 or SEQ ID NO: 4,and which retain drimenol synthase activity when overexpressed or ectopically expressed in a plant. Polypeptide sequences that are at least 55% identical to the polypeptides of the present invention are considered sufficiently identical.

Homologous sequences may be derived from any plants including monocots or dicots, and especially crops including but not limited to tomato, pepper, eggplant, lettuce, sunflower, oilseed rape, broccoli, cauliflower and cabbage crops, cucumber, melon, watermelon, pumpkin, squash, peanut, soybeans, cotton, beans, avocado, onion, endive, leek, roots such as arrowroot, carrot, beet, turnip, radish, yam, cassava, potatoes, sweet potatoes and okra. Homologous sequences may also be derived from crop species including maize, barley, pearl millet, wheat, rye, sorghum, rice, tobacco and forage grasses. Homologous sequences may be derived from tree species and fleshy fruit species such as lemons, tangerines, oranges, grapes, peaches, plums, currant, cherries, melons, strawberry, and mango, or from ornamental plant species such as hibiscus, poinsettia, lily, iris, rose and petunia, and the like. Additionally, homologous sequences may be derived from plant species that are wild relatives of crop plant species. For example, homologous sequences may be derived from nightshade Atropa belladonna which is a wild relative of a cultivated tomato Solanum lycopersicum, or teosinte species related to maize.

Homologous sequences include orthologous or paralogous sequences. Methods of identifying orthologs or paralogs including phylogenetic methods, sequence similarity and hybridization methods are known in the art and are described herein.

Paralogs result from gene duplication that gives rise to two or more genes with similar sequences and similar functions. Paralogs typically cluster together and are formed by duplications of genes within related plant species. Paralogs are found in groups of similar genes using pair-wise Blast analysis (Feng and Dollitle, 1987 J Mol Evol: 25:351) or during phylogenetic analysis of gene families using programs such as CLUSTAL (Thompson et al. 1994 Nucl Acid Res 22:4573; Higgins et al., 1996 Methods Enzymol 266:383). In paralogs, consensus sequences can be identified characteristic to sequences within related genes and having similar functions of the genes.

Orthologs, or orthologous sequences, are sequences similar to each other because they are found in species that descended from a common ancestor. For instance, plant species that have common ancestors are known to contain many enzymes that have similar sequences and functions. The skilled artisan can identify orthologous sequences and predict the functions of the orthologs, for example, by constructing a polygenic tree for a gene family of one species using CLUSTAL or BLAST programs. A method for identifying or confirming similar functions among homologous sequences is by comparing of the transcript profiles in plants overexpressing or lacking (in knockouts/knockdowns) related polypeptides. The skilled person will understand that genes having similar transcript profiles, with greater than 50% regulated transcripts in common, or with greater than 70% regulated transcripts in common, or greater than 90% regulated transcripts in common will have similar functions. Homologs, paralogs, orthologs and any other variants of the sequences herein are expected to function in a similar manner by making plants producing drimenol synthase proteins.

An embodiment of the invention provides amino acid sequences of drimenol synthase proteins including orthologs and paralogs as well as methods for identifying and isolating orthologs and paralogs of the drimenol synthases in other organisms. Preferably, so identified orthologs and paralogs of the drimenol synthase retain drimenol synthase activity and are capable of producing drimenol starting from FPP precursors.

In yet another embodiment, a “variant” or “derivative” of the polypeptide set forth in SEQ ID N0:2 or SEQ ID N0:4 is provided, such variant or derivative being a polypeptide comprising an amino acid sequence with substantial similarity to that of the polypeptide herein, e.g. being at least 70%, 75%, 80%, 85%, 90%, 92%, 95%, 96%, 97%, 98% or 99% identical to SEQ ID N0:2 or SEQ ID N0:4, preferably over its full length. The amino acid sequences of the polypeptide of the invention and variants thereof may differ by deletions, additions, and/or substitutions of amino acids while retaining functional equivalence to the polypeptide (i.e. drimenol synthesis starting from FPP precursors). For instance, amino acids of the polypeptide of the invention may be modified based on similarity in hydrophobicity, hydrophilicity, solubility, polarity of amino acid residues, as long as the variant polypeptide remains functionally equivalent (i.e. drimenol synthesis starting from FPP precursors) to the polypeptide of the invention.

Variants also include proteins having drimenol synthase activity, which have been derived, by way of one or more amino acid substitutions, deletions or insertions, from the polypeptide having the amino acid sequence of SEQ ID NO:2 or SEQ ID NO 4. Preferably, such proteins comprise from 1, 2, 3, 4, 5, 6, 7, 8, 9, 10 or more up to about 100, 90, 80, 70, 60, 50, 45, 40, 35, 30, 25, 20, 15 amino acid substitutions, deletions or insertions.

A variant may also differ from the polypeptide of the invention by attachment of modifying groups which are covalently or non-covalently linked to the polypeptide backbone. The variant also includes a polypeptide which differs from the polypeptide of the present invention by introduced N-linked or 0-linked glycosylation sites, and/or an addition of cysteine residues. The skilled artisan will recognise how to modify an amino acid sequence and preserve biological activity.

The functionality or activity of any drimenol synthase protein, variant or fragment, may be determined using various methods. For example, transient or stable overexpression in plant, bacterial or yeast cells can be used to test whether the protein has activity, i.e., produces drimenol from FPP precursors. Drimenol synthase activity may be assessed in a yeast expression system, such as the assay described in Example 2 herein on the production of drimenol, indicating functionality. A variant or derivative of a drimenol synthase polypeptide of the invention retains an ability to produce drimenol from FPP precursors. Amino acid sequence variants of the drimenol synthases of the present invention may have additional desirable biological functions including, e.g., altered substrate utilization, reaction kinetics, product distribution or other alterations.

An embodiment herein provides polypeptides of the invention to be used in a method to produce drimenol or at least one drimenol derivative by contacting an FPP precursor with the polypeptides of the invention either in vitro or in vivo.

To carry out the in vitro method, the polypeptide having the amino acid sequence of SEQ ID NO:2 or SEQ ID NO:4, or variants, or fragments thereof, e.g., those that will possess at least 70%, 75%, 80%, 85%, 90%, 92%, 95%, 96%, 97%, 98% or 99% identity over the entire length of the polypeptides of the invention, can be isolated from any organism expressing the same, for instance, after transformation with the polynucleotides of the invention.

It is known in the art that a cell can be transformed with a nucleic acid encoding a desired protein to be secreted, for instance, to the culture medium, so as to produce large quantities of the protein. The protein can be collected from the culture medium and further used, for example, to produce drimenol or a drimenol derivative.

In vitro or in vivo produced drimenol can be further used for synthesis of many drimenol derivatives (Cortes et al. 2011 Nat Product Communications 6: 477). For instance, the list of compounds that was synthesized from drimenol includes without limitation driman-8-ol, driman-8,11-diol, drim-8-en-7-one, forskolin, cinnamodial, (+)-albicanol, (−)-uvidin, (+)-isopolygonal, (−)-polygodial, (−)-ugandensidial, (−)-warburganal, ambergris, drimenal, drimenoic acid, isodrimenin, cinnamolide, confertolin, confertifolin, drimendiol, and polygodial acid.

Additionally, drimenol so produced can be used as a starting compound for synthesis of ambergris and ambergris related compounds, that previously was obtained from nearly exterminated sperm whales and has long been used by perfumers for fragrance properties and unique fixative powers. Nitrogenated drimenol derivatives obtained by binding functional groups to (−)-drimenol at C-11, may possess an increased antifungal activity. In general, drimenol may be used to synthesize drimenol derivatives possessing fungicidal, insecticidal, fragrance, antifeedant, and/or food taste modifying properties.

In an alternative embodiment, the cell or cells are engineered to accumulate the polypeptides of the invention within the cell. The skilled person will recognize how to extract proteins or polypeptides from the cell, for instance, by using the following techniques: repeated freezing and thawing, sonication, homogenization by high pressure, filtration, or permeabilization by organic solvents, and the like. After the extraction, proteins can be re-suspended in a buffer solution at optimal pH and then FPP may be added to the solution to produce drimenol. After incubation, the resultant drimenol may be removed from the solution by standard isolation and purification procedures.

It may be particularly advantageous to direct the localization of the drimenol synthase proteins to a subcellular compartment, for example, to plastids, preferably chloroplasts, mitochondria, endoplasmic reticulum, or vacuoles. Targeting of proteins to the particular cell structure may provide most efficient functioning for the desired expressed proteins.

It is well known in the art that proteins can be directed to the chloroplast by including at their N-terminus a chloroplast transit peptide. Naturally occurring chloroplast targeted proteins, synthesized as larger precursor proteins containing an N-terminal chloroplast targeting peptide directing the precursor to the chloroplast import machinery, have been previously identified (Hesse et al. 1989, EMBO J 8: 2453; Klosgen et al. 1989 Mol Gen Genet 217: 155; Klosgen and Weil 1991 Mol Gen Genet 225: 297; Shcherban et al. 1995 Proc Natl Acad Sci USA 92: 9245; Park et al. 1997 J Biol Chem 272: 6876; Tavladoraki et al. 1998 FEBS Lett. 426: 62; Neuhaus and Rogers 1998 Plant Mol Biol 38: 127; Bih et al. 1999, J Biol Chem 274: 22884; Morris et al. 1999 Biochem Biophys Res Commun 255: 328; Terashima et al. 1999 Appl Microbiol Biotechnol 52: 516).

Chloroplast targeting peptides have been found particularly useful for designing plants with overproduction of terpenes which were observed to be toxic to the host if overexpressed in cytosol but not in plastids.

For this purpose, in certain embodiments the chimeric genes of the invention comprise a coding region encoding a signal or target peptide, linked to the drimenol synthase protein coding region of the invention.

Examples of sequences encoding peptides which are suitable for directing the targeting of the drimenol synthase gene product to the plastid or the chloroplast of the plant cell include the transit peptide of ferredoxin-NADP+oxidoreductase from spinach (Oelmuller et al. 1993 Mol Gen Genet 237:261) and the like (see U.S. Pat. No. 5,635,618; Wong et al. 1992 Plant Mol Biol 20:81; PCT patent application WO 00/26371).

Examples of sequences which are suitable for directing the targeting of the drimenol synthase gene product to mitochondria such as Cox IV targeting signal (Kohler et al. 1997 Plant J 11: 61) are also within the scope of the invention herein.

To allow secretion of the drimenol synthase proteins to the outside of the transformed host cell, an appropriate secretion signal peptide may be fused to the amino terminal end, e.g., N-terminal end, of the drimenol synthase protein. Putative transit peptides can be detected using computer based analysis, using programs such as the program Signal Peptide search (SignalP V3.0; Von Heijne, Gunnar, 1986 and Nielsen et al. 1996).

Also preferred are peptides targeting secretion of a protein linked to such peptide outside the cell, such as the secretion signal of the potato proteinase inhibitor II (Keil et al. 1986 Nucl Acids Res 14: 5641), the secretion signal of the alpha-amylase 3 gene of rice (Sutliff et al. 1991 Plant Mol Biol 16:579) and the secretion signal of tobacco PR1 protein (Cornelissen et al. 1986 EMBO J 5:37).

Particularly useful transit peptides in accordance with the invention include a chloroplast transit peptide (Van Den Broeck et al. 1985 Nature 313:358), or an optimized chloroplast transit peptide (U.S. Pat. No. 5,510, 471 and U.S. Pat. No. 5,635, 618) causing transport of the protein to the chloroplasts, a secretory signal peptide or a peptide targeting the protein to other plastids, mitochondria, the ER, or another organelle.

Besides targeting polypeptides of the invention to intracellular organelles, methods of transformation of the plastid genome, preferably chloroplast genome, or mitochondrial genome are also included in the invention. Transformation of organelles is known to the skilled artisan and provides means to control environmental transgene spread (Sidorov et a., 1999 Plant J 19: 209; Lutz et al. 2004 Plant J 37: 906).

### Polynucleotides of the Invention

The present invention also provides an isolated, recombinant or synthetic polynucleotide encoding a polypeptide or variant polypeptide of the present invention.

An embodiment of the invention provides an isolated, recombinant or synthetic nucleic acid sequence of SEQ ID NO:1, or a variant thereof which is at least 70%, 75%, 80%, 85%, 90%, 92%, 95%, 96%, 97%, 98% or 99% identical to a nucleic acid sequence of SEQ ID

NO:1 which encodes a drimenol synthase having the amino acid sequence as shown in SEQ ID NO:2 or a nucleic acid sequence of SEQ ID NO:3 which encodes a drimenol synthase having the amino acid sequence of SEQ ID NO:4, or fragments thereof that catalyze production of drimenol in a cell from a FPP precursor. Embraced by the present invention are cDNA, genomic DNA and RNA sequences. Any nucleic acid sequence encoding the drimenol synthase or variants thereof is referred herein as a drimenol synthase encoding sequence.

According to a preferred embodiment, the nucleic acid of SEQ ID NO:1 is the coding sequence of a drimenol synthase gene encoding the drimenol synthase obtained from V. officinalis as described in the Examples herein.

In yet another embodiment the nucleic acid of SEQ ID NO:3 derived from P. hydropiper and coding for a drimenol synthase protein is also provided.

A fragment of a polynucleotide of SEQ ID NO:1 or SEQ ID NO:3 refers to contiguous nucleotides that is preferably at least 15 bp, at least 30 bp, at least 40 bp, at least 50 bp and/or at least 60 bp in length of the polynucleotide of the invention herein. Preferably the fragment of a polynucleotide comprises at least 25, more preferably at least 50, more preferably at least 75, more preferably at least 100, more preferably at least 150, more preferably at least 200, more preferably at least 300, more preferably at least 400, more preferably at least 500, more preferably at least 600, more preferably at least 700, more preferably at least 800, more preferably at least 900, more preferably at least 1000 contiguous nucleotides of the polynucleotide of the invention. Without being limited, the fragment of the polynucleotides herein may be used as a PCR primer, and/or as a probe, or for anti-sense gene silencing or RNAi.

It is clear to the person skilled in the art that genes, including the polynucleotides of the invention, can be cloned on basis of the available nucleotide sequence information, such as found in the attached sequence listing, by methods known in the art. These include e.g. the design of DNA primers representing the flanking sequences of such gene of which one is generated in sense orientations and which initiates synthesis of the sense strand and the other is created in reverse complementary fashion and generates the antisense strand. Thermo stable DNA polymerases such as those used in polymerase chain reaction are commonly used to carry out such experiments. Alternatively, DNA sequences representing genes can be chemically synthesized and subsequently introduced in DNA vector molecules that can be multiplied by e.g. compatible bacteria such as e.g. E. coli.

In a related embodiment of the invention, PCR primers and/or probes for detecting nucleic acid sequences encoding a drimenol synthase are provided. The skilled artisan will be aware of methods to synthesize degenerate or specific PCR primer pairs to amplify a nucleic acid sequence encoding the drimenol synthase or fragments thereof, based on SEQ ID NO:1 or SEQ ID NO:3. A detection kit for nucleic acid sequences encoding the drimenol synthase may include primers and/or probes specific for nucleic acid sequences encoding the drimenol synthase, and an associated protocol to use the primers and/or probes to detect nucleic acid sequences encoding the drimenol synthase in a sample. Such detection kits may be used to determine whether a plant has been modified, i.e., transformed with a sequence encoding the drimenol synthase.

Due to the degeneracy of the genetic code, more than one codon may encode the same amino acid sequence, multiple nucleic acid sequences can code for the same protein or polypeptide. Where appropriate, the nucleic acid sequences encoding the drimenol synthase may be optimized for increased expression in the host cell. For example, nucleotides of the invention may be synthesized using codons preferred by a host for improved expression (See Campbell and Gowri 1990 Plant Physiol 92:1; Bennetzen and Hall 1982 J Biol Chem 257:3026). Methods are available in the art for constructing plant-preferred synthetic DNA sequences (see U.S. Pat. Nos. 5,380,831 and 5,436,391). Codon usage tables for plant species are publicly available (see Ikemura 1993 In Plant Molecular Biology Labfax, Croy ed., Bios Scientific Publishers Ltd, pp. 37-48; Codon Usage Database at the Kazusa DNA Research Institute, Japan).

The nucleic acid sequences obtained by mutations of SEQ ID NO:1 and SEQ ID NO:3 can be routinely made and are also within embodiments of the present invention. It is clear to the skilled artisan that mutations, deletions, insertions, and/or substitutions of one or more nucleotides can be introduced into the DNA sequence of SEQ ID NO:1 or SEQ ID NO:3. Generally, a mutation is a change in the DNA sequence of a gene that can alter the amino acid sequence of the polypeptide produced.

To test a function of variant DNA sequences according to the invention, the sequence of interest is operably linked to a selectable or screenable marker gene and expression of the reporter gene is tested in transient expression assays with protoplasts or in stably transformed plants. The skilled artisan will recognize that DNA sequences capable of driving expression are built as modules. Accordingly, expression levels from shorter DNA fragments may be different than the one from the longest fragment and may be different from each other. Embraced by the present invention are also functional equivalents of the nucleic acid sequence coding the drimenol synthase proteins of the present invention, i.e., nucleotide sequences that hybridize under stringent conditions to the nucleic acid sequence of SEQ ID NO:1 or SEQ ID NO:3.

A stringent hybridization is performed at a temperature 65° C. and most preferably at 55° C. in double strength (2×) citrate buffered saline (SSC) containing 0.1% SDS followed by rinsing of the support at the same temperature but with a buffer having reduced SSC concentration. Such reduced concentration buffers are typically one tenth strength SSC (0.1×SSC) containing 0.1% SDS, preferably 0.2×SSC containing 0.1% SSC and most preferably half strength SSC (0.5×SSC) containing 0.1% SDS. Functional equivalents of the drimenol synthase proteins from other organism can be found by hybridizing a nucleic acid sequence with SEQ ID NO:1 or SEQ ID NO:3 with genomic DNA isolated from other organisms.

The skilled artisan will be aware of methods to identify homologous sequences in other organisms and methods (identified in the Definition section herein) to determine the percentage of sequence identity between homologous sequences. Such newly identified DNA molecules then can be sequenced and the sequence can be compared with the nucleic acid sequence of SEQ ID NO:1 or SEQ ID NO:3 and tested for functional equivalence. Within the scope of the present invention are DNA molecules having at least 75%, preferably 80%, more preferably 90% and most preferably 95% or more sequence identity to the nucleotide sequence of SEQ ID NO:1 or SEQ ID NO:3.

A related embodiment of the invention provides a nucleic acid sequence which is complementary to the nucleic acid sequence according to SEQ ID NO:1 or SEQ ID NO:3, such as inhibitory RNAs, or nucleic acid sequence which hybridizes under stringent conditions to at least part of the nucleotide sequence according to SEQ ID NO:1 or SEQ ID NO:3.

An alternative embodiment of the invention provides a method to alter gene expression in a host cell. For instance, the polynucleotide of the invention may be enhanced or overexpressed or induced in certain contexts (e.g. following insect bites or stings or upon exposure to a certain temperature) in a host cell or host organism.

Alteration of expression of a polynucleotide of the present invention also results in “ectopic expression” which is a different expression pattern in an altered and in a control or wild-type organism. Alteration of expression occurs from interactions of polypeptide of the invention with exogenous or endogenous modulators, or as a result of chemical modification of the polypeptide. The term also refers to an altered expression pattern of the polynucleotide of the invention which is altered below the detection level or completely suppressed activity.

Overexpression occurs when a gene encoding the drimenol synthase of the invention is under control of a strong constitutive or a tissue specific promoter. For example, strong constitutive promoters include constitutive 35S or enhanced 35S cauliflower mosaic virus (CaMV) promoters, maize ubiquitin promoter, rice actin promoter, the emu promoter, and the like. Tissue specific promoters include without limitation leaf-preferred, trichome-specific, root-preferred, epidermis-preferred or promoters specific to reproductive tissues such as pollen, such that the drimenol synthase gene under control of the tissue specific promoter is expressed only in specific tissues or organs and/or during certain developmental stages.

As the constitutive expression of a drimenol synthase protein may be detrimental to plant cell or may result in lower yield, an embodiment of the invention herein provides inducible promoters. Examples of inducible promoters include wound-inducible promoters, temperature inducible promoters (U.S. Pat. No. 5,447,858), chemically inducible promoters, promoters inducible by light, by anaerobic conditions (ADH1S), by pathogens (EP 759,085) or by senescence (U.S. Pat. No. 5,689,042), or other inducible promoters.

In a preferable embodiment, an insect-inducible promoter is used such that the drimenol synthase protein will be produced only in plants wounded by pest insects. Even more preferable, the promoter is inducible by a broad range of pest insects. Alternatively, a host plant may include more than one gene encoding the drimenol synthase protein each under control of a different pest inducible promoter to ensure that the drimenol synthase protein is produced following attacks by different pest insects. Examples of such inducible promoter are known to the skilled person and include, but are not limited to the potato proteinase inhibitor II (pinII)-promoter (Godard et al. 2007 Plant Cell Reports 26(12):2083), tobacco WIPK promoter (Seo et al. 1999 Plant Cell 11:289), LOX (e.g. in in Arabidopsis, tomato, tobacco), or the Wir1 family promoter (e.g. in wheat; Yuan et al. 2004 J Plant Physiol 161(1):79).

In yet another preferable embodiment, the method for producing drimenol and/or at least one drimenol derivative is carried out in vivo. For instance, drimenol may be produced using the method of the invention herein when the host cell is naturally capable of producing FPP and one or more stereoisomers thereof. Alternatively, the host cell that does not produce FPP naturally or produces it in a low amount may be engineered to produce FPP or to increase the amount FPP compared to a non-engineered host cell. Alternatively, the skilled artisan would recognize how to achieve overexpression of the substrate of the drimenol synthase protein (i.e., FPP) to produce drimenol and/or drimenol derivatives. For example, the host cell may be transformed with exogenous nucleotides encoding the farnesyldiphosphate synthase to produce FPP in an organism. Co-expressing drimenol synthase with farnesyl diphosphate synthase (FPS) and 3-hydroxy-3-methylglutaryl-CoA reductase (HMGR) can increase the flux of sesquiterpene precursors to drimenol synthase: FPS supplies the direct precursor of drimenol, i.e., FPP, while HMGR is considered the most important rate-limiting step in the mevalonate pathway.

For production of drimenol in vivo, the host cell capable of producing FPP may be transformed with the nucleic acid encoding the drimenol synthase having the amino acid sequence of SEQ ID NO:2 or SEQ ID:4, or having at least 70%, 75%, 80%, 85%, 90%, 92%, 95%, 96%, 97%, 98% or 99% identity over the entire length of the polypeptides of the invention,in such a way so to enable expression of the polypeptide within the host cell. The host cell or organism transformed with the polynucleotides of the invention is further cultured under conditions permitting production of drimenol and/or drimenol derivatives. For example, a culture medium may be selected to enable culture and/or differentiation of the transformed cell to enable synthesis of drimenol and/or drimenol derivatives in the cells or the resultant transformed organism.

Conversion of drimenol into antifeedant drimenol derivatives such as polygodial or warburganal or other drimane derivatives may be achieved by co-expression in vivo of one or more cytochrome P450 enzymes. These enzymes are known to be capable of carrying out additional hydroxylations and oxidations of the sesquiterpene backbone. Genes for such enzymes may be isolated from the tissues of P. hydropiper or V. officinalis which are rich in such derivatives.

In one embodiment, several drimenol synthase encoding nucleic acid sequences are co-expressed in a single host, preferably under control of different promoters. Alternatively, several drimenol synthase protein encoding nucleic acid sequences can be present on a single transformation vector or be co-transformed at the same time using separate vectors and selecting transformants comprising both chimeric genes. Similarly, one or more drimenol synthase encoding genes may be expressed in a single plant together with other chimeric genes, for example encoding other proteins which enhance insect pest resistance, or others.

It is understood that different proteins can be expressed in the same plant, or each can be expressed in a single plant and then combined in the same plant by crossing the single plants with one another. For example, in hybrid seed production, each parent plant can express a single protein. Upon crossing the parent plants to produce hybrids, both proteins are combined in the hybrid plant.

The nucleic acid sequences of the invention encoding drimenol synthase proteins can be inserted in expression vectors and/or be contained in chimeric genes inserted in expression vectors, to produce drimenol synthase proteins in a host cell or host organism. The vectors for inserting transgenes into the genome of host cells are well known in the art and include plasmids, viruses, cosmids and artificial chromosomes. Binary or co-integration vectors into which a chimeric gene is inserted are also used for transforming host cells.

An embodiment of the invention provides recombinant expression vectors comprising a nucleic acid sequence of a drimenol synthase gene, or a chimeric gene comprising a nucleic acid sequence of a drimenol synthase gene, operably linked to associated nucleic acid sequences such as, for instance, promoter sequences. For example, a chimeric gene comprising a nucleic acid sequence of SEQ ID NO:1 or SEQ ID NO:3 may be operably linked to a promoter sequence suitable for expression in plant cells, bacterial cells or fungal cells, optionally linked to a 3′ non-translated nucleic acid sequence.

Alternatively, the promoter sequence may already be present in a vector so that the nucleic acid sequence which is to be transcribed is inserted into the vector downstream of the promoter sequence. Vectors are typically engineered to have an origin of replication, a multiple cloning site, and a selectable marker.

Expression vectors for use with bacterial, fungal, yeast and mammalian cell hosts are described, for instance, in Pauwels et al. Cloning vectors, A Laboratory Manual, 1985 Elsevier, N.Y. and Sambrook et al. Molecular Cloning: A Laboratory Manual, 2^{nd} ed. 1989, Cold Spring Harbor Laboratory Press. Cell-free translation systems may be also employed to produce the proteins of the invention herein using RNAs derived from nucleic acid sequences of the present invention.

The vector that is used to transform the host cells and the chimeric gene is preferably inserted in the nuclear genome or into the genome of cell organelles, i.e., mitochondria or plastids, such that the expression of the nucleic acid sequence is driven by the activity of the promoter. See Arabidopsis, A Laboratory Manual Eds. Weigel and Glazebrook, Cold Spring Harbor Laboratory Press (2002) and Maniatis et al., Molecular Cloning, Cold Spring Harbor Laboratory Press (1982).

In the invention herein, the host cell or host organism may be a non-producer of drimenol. A vector such as an expression vector containing a drimenol synthase gene or a chimeric gene containing a drimenol synthase nucleotide sequence may be introduced into such a host cell or host organism such that when the gene product is expressed the host cell or host organism is capable of producing drimenol and/or drimenol derivatives. The host cell is cultivated in a cell culture under conditions to bring about expression of a drimenol synthase gene and, upon contacting the FPP precursor, recovering drimenol and/or drimenol derivatives from the cell culture.

The host cell may be selected from a group of prokaryotic or eukaryotic cells. Suitable prokaryotic cells include gram-positive and gram-negative bacteria such as Escherichia coli and Agrobacterium tumefaciens.

Alternatively, proteins of the invention herein may be expressed in eukaryotic cells. For example, nucleic acid sequences (or fragments thereof) encoding drimenol synthase proteins may be used to transform fungal cells, mammalian cells, plant cells or optionally non-human cells. Yeast host cells, for instance, belonging to genus Saccharomyces (e.g., S. cerevisiae), Pichia or Kluyveromyces, or other yeast genera, may be also employed to express proteins of the present invention. The altered cell may give rise to a tissue or a whole organism. Suitable hosts may further include algae, or insects.

In a preferable embodiment, a host includes a plant. Any plant may be a suitable host including dicotyledonous plants (dicots) or monocotyledonous plants (monocots). Plants suitable for expression of a polypeptide of the invention include, but are not limited to crop species, which are natural hosts of insect pests such as tomato, pepper, eggplant, lettuce, sunflower, oilseed rape, broccoli, cauliflower and cabbage crops, cucumber, melon, watermelon, pumpkin, squash, peanut, soybeans, cotton, beans, cassava, potatoes, sweet potato and okra. Crop species also include maize, barley, pearl millet, wheat, rye, sorghum, rice, and forage grasses.

Additionally, plant hosts include tree species, fleshy fruit species such as grapes, peaches, plums, strawberry and mango, and ornamental species such as hibiscus, poinsettia, lilies, iris, rose and petunia.

Especially preferred are plants belonging to the family Solanaceae, including plants that belong to the genera Solanum, Capsicum, Nicotiana, Petunia, and the like. In a preferred embodiment, vegetable species especially of genus Solanum are included, for example, tomato (S. lycopersicum), eggplant (S. melongena), pepino (S. muricatum), and the like.

The skilled artisan is aware of the methods to modify host cells or non-human organisms. Transformation of bacteria is known in the art and can be carried out, for example, using the electroporation technique. The codon usage of a nucleic acid sequence may be optimized for expression in prokaryotic cells. Other optimizations of expression in prokaryotic cell known to the skilled artisan include, for instance, removal of intron sequences. The present invention also embraces the methods for genetic modification of yeast, fungal and preferably plant cells.

Methods for genetic modification of plants include, without limitation, Agrobacterium-mediated transformation of plant explants and direct gene transfer to protoplasts, pollen, e.g. by electroporation or by using polyethylene glycol, injection into reproductive cells, organs and immature embryos, plastid, chloroplast and mitochondria transformation, gene transfer by particle bombardment. Other gene delivery devices known to the skilled artisan include lipid and viral vectors, electroporation, agitation with silicon carbide whiskers, and chemical methods.

Methods for transforming dicots have been published, for instance, for tobacco, tomato, potato, pepper, soybean, Brassica, cotton watermelon, melon, strawberry, mint and other dicots. Transformation of monocots using Agrobacterium, particle bombardment, and electroporation has also been reported, for example, for maize, barley, rice, oat, sugar cane, wheat, rye, tall fescue, and other monocots.

Agrobacterium-mediated transformation is a preferred method to introduce the nucleic acid molecule of the invention into plant explants. Agrobacterium tumefaciens harbors a natural vector called the Ti plasmid, which was engineered to make it suitable for introduction of exogenous nucleic acid molecules into plant genomes. For genetic transformation, plant-derived explants are incubated with suspension of Agrobacterium cells followed by cultivation of the explants on the medium containing a selective agent that promotes growth and regeneration of the transformed cells only.

In this regard, in a preferred embodiment of the invention herein, a T-DNA vector that includes a nucleic acid sequence encoding a drimenol synthase protein, or a chimeric gene containing a nucleic acid sequence encoding a drimenol synthase protein, inserted in Agrobacterium tumefaciens can be used to stably transform the plant cell. The construction of T-DNA vectors for Agrobacterium-mediated transformation is well-known in the art. Within the T-DNA vector, a drimenol synthase nucleic acid sequence of SEQ ID NO:1 or SEQ ID NO:3, or a chimeric gene containing a drimenol synthase nucleic acid sequence of SEQ ID NO:1 or SEQ ID NO:3, operably linked to promoter is located between T-DNA border sequences.

The gene encoding the drimenol synthase is inserted into the plant genome such that the coding sequence of the gene is located upstream of a suitable 3′end untranslated region (3′UTR). Suitable 3′ends include, without limitation, those of the CaMV 35S gene, the nopaline synthase gene, the octopine synthase gene and the like.

Each of the methods has advantages and disadvantages, and therefore one particular method suitable for transformation of one organism may not be effective for another organism, but the skilled artisan will be informed of which method of transformation to use for a particular organism. Protocols for selection and regeneration of transformed plants are well known in the art and the skilled person will select an appropriate protocol to recover transformed plants at high frequency.

In one embodiment, the nucleic acid sequence encoding the drimenol synthase protein can be stably integrated into the nuclear genome of a plant cell so that such plant cell may be cultivated and used to produce drimenol and/or drimenol derivatives.

An alternative embodiment of the invention provides a method for producing a transgenic plant capable of producing drimenol and/or increased levels of drimenol or a transgenic plant having an enhanced insect resistance compared to a non-transgenic plant of a similar genetic background, including the following steps: transforming a plant cell with a nucleic acid sequence encoding a protein having drimenol synthase activity and comprising the amino acid sequence of SEQ ID NO:2 or SEQ ID NO:4 or an amino acid sequence that is at least 70%, 75%, 80%, 85%, 90%, 92%, 95%, 96%, 97%, 98% or 99% identical to the amino acid sequence of SEQ ID NO:2 or SEQ ID NO:4, or, with a chimeric gene comprising a nucleic acid sequence encoding a protein having drimenol synthase activity and comprising the amino acid sequence of SEQ ID NO:2 or SEQ ID NO:4 or an amino acid sequence that is at least 70%, 75%, 80%, 85%, 90%, 92%, 95%, 96%, 97%, 98% or 99% identical to the amino acid sequence of SEQ ID NO:2 or SEQ ID NO:4, operably linked to a promoter, and regenerating a plant. An embodiment of the invention herein also provides a genetically engineered plant cell that includes a nucleic acid sequence set forth in SEQ ID NO:1 or SEQ ID NO:3, or with a chimeric gene comprising a nucleic acid sequence as set forth in SEQ ID NO:1 or SEQ ID NO:3, and a plant regenerated from the cell. A genetically engineered plant of the invention includes the plant having the capacity to produce drimenol and/or drimenol derivatives, or a plant having the capacity to produce an enhanced level of drimenol and/or drimenol derivatives, compared to a non-genetically engineered plant of the same genetic background.

Single copy transformant plants can be identified using Southern Blot analysis or PCR based methods. Alternatively, drimenol, or drimenol derivatives may be determined by analytical methods including, for instance, gas chromatography-mass spectrometry (GC-MS).

The resulting transformed plant can be crossed or selfed and used for plant breeding production of a population of transformed plants producing the proteins of the invention.

The method may also include the step of screening the transgenic plant or a plant derived from the transgenic plant by selfing or crossing, for resistance to one or more insect pest and identifying a plant having an enhanced resistance to at least one insect pest, for production of drimenol and/or drimenol derivatives, and/or identifying a plant having the capacity to produce drimenol and/or drimenol derivatives, or a plant having the capacity for enhanced drimenol production and/or drimenol derivatives.

A related embodiment of the invention also provides for a tissue culture obtained from the transgenic plant such that the culture has enhanced production or secretion of drimenol and/or drimenol derivatives, particularly as compared to a non-transgenic plant of the same genetic background, and a method for isolating drimenol and/or drimenol derivatives from the tissue culture of the invention.

### Insects Controlled by (Over) Expressing Drimenol Synthase in Plants

An embodiment of the invention provides a method for providing and/or increasing resistance to a pest insect by (over) expressing the genes encoding drimenol synthase.

“Insect pest resistance” is an enhanced ability of modified transgenic plants of the present invention to withstand attacks of one or more pests compared to wild type or control plants. Methods to assess insect pest resistance in plants are known in the art. For example, disease symptoms may be scored visually at one or more time points after infestation or contact with an insect pest. Alternatively, insect pests may be detected and optionally quantified during infestation of the plants in an assay. A modified plant shows enhanced pest resistance if the number of insect pests detected in the tissue is significantly lower compared to the number of insects detected in control. Preferably, a significant increase in average yield of modified plants of the present invention compared to control (e.g. at least 1%, 2%, 5%, 10% or more) when plants are grown under insect pest pressure provides an indirect measurement of enhanced resistance to pest insects. Statistical analyses are employed to determine existence of significant differences.

The invention now having been fully described it is exemplified in the Examples below and in the claims, which are not to be construed as further limiting. References cited herein are hereby incorporated by reference in their entireties.

### BRIEF DESCRIPTION OF THE DRAWINGS

FIG. 1 shows a cDNA sequence of SEQ ID NO:1 encoding drimenol synthase from V. officinalis including translated sequence of SEQ ID NO:2.

FIG. 2 shows a cDNA sequence of SEQ ID NO:3 encoding drimenol synthase from P. hydropiper including translated sequence of SEQ ID NO:4.

FIG. 3 depicts an alignment of the drimenol synthase amino acid sequences from V. officinalis (DSval) (SEQ ID NO:2) and P. hydropiper (DSph) (SEQ ID NO:4). Amino acid sequences were aligned with ClustalW. Identical and similar residues are underlined and in bold letters, respectively.

FIG. 4 depicts a set of chromatograms and mass spectra profiles showing the results of Gas Chromatography-Mass Spectrometry (GC-MS) analysis performed on the yeast strain WAT11 expressing drimenol synthase from either V. officinalis (DSval) or P. hydropiper (DSph). Panels A-D show chromatograms of the n-dodecane layer from the yeast strain WAT11. In the drawings, relative abundance of ions (with respect to ions of highest abundance) is shown as function of time (minutes). Note that the y-axis scales of the chromatograms are not identical. Specifically, panel A shows the drimenol standard (retention time (rt)=17.7 min). Panel B shows that yeast cells transformed with a gene encoding drimenol synthase from V. officinalis (DSval) produce drimenol. The size of the peak generated by drimenol (rt=17.17 min) matches the size of the peak generated by an empty vector in control treatment (rt=17.92 min) indicating low relative abundance of drimenol ions. Panel C shows that yeast cells transformed with the gene encoding the drimenol synthase from P. hydropiper (DSph) produce drimenol. The drimenol synthase from P. hydropiper was observed to have a strong enzymatic activity judging by the size of the drimenol peak, rt=17.17 min, that significantly exceeds the size of the peak generated in control treatment with an empty vector, rt=17.92 min. Panel D shows that yeast cells transformed with an empty vector produce a peak identifiable at 17.92 min. Furthermore, panels E-G show that the mass spectra profiles for the drimenol standard (panel E; rt=17.17 min) match the mass spectra profiles for drimenol produced by yeast cells after transformation with each of drimenol synthase proteins from V. officinalis (DSval) (panel F) and P. hydropiper (DSph) (panel G).

FIG. 5 depicts a set of chromatograms of dichloromethane extracts of leaves from Nicotiana benthamiana infiltrated with an expression vector containing a gene encoding drimenol synthase from V. officinalis under control of the 35S promoter (35S-DSval) with mitochondrial targeting (panel C), the drimenol standard (panel B) and an empty vector control construct (panel A). The y-axis scales of the chromatograms are identical.

FIG. 6 displays a set of chromatograms of dichloromethane extracts of leaves from Nicotiana benthamiana infiltrated with an expression vector containing a gene encoding the drimenol synthase from P. hydropiper under control of rbcS1 promoter (RBCS-DSph) with plastid (panel C) or cytosolic (panel D) targeting, the drimenol standard (panel A) and a control empty vector construct (panel B). Note that the y-axis scales of the chromatograms are not identical.

### EXAMPLES

### Example 1

### Identification of the Drimenol Synthase Genes

The identification of the drimenol synthase (DS) gene from V. officinalis relied first on preparing mRNA from root tissues. RNA prepared from V. officinalis roots was used to prepare cDNA by standard protocols. This cDNA was subsequently used as a template to generate terpene synthase gene fragments. The sense primer 1s (5′-GAY GAR AAY GGI AAR TTY AAR GA-3′) and antisense primer 2as (5′-CC RTA IGC RTC RAA IGT RTC RTC-3′) were used using Super Taq polymerase based on a program of 30 sec 94° C., 1 min 42 C, and 1 min 72 C for 35 cycles. The PCR fragments were ligated into pGEM-T Easy vector and transformed to competent E. coli cells. Ampicillin resistant colonies were picked, grown overnight and plasmid DNA was isolated by standard protocols. Plasmid DNA containing the correct insert was sequenced and based on the obtained sequences primers were designed for 5′and 3′ RACE PCR to obtain the full length sequence. The complete coding region of the cDNA was amplified based on two DNA oligonucleotides (5′-ATGTCTACTGCATTAAACAG-3′ and 5′-TCTATACGGGGACGGGGTC-3′) homologous to the 5 and 3′regions of the gene. The identification of drimenol synthase gene relied first on obtaining a 454 EST library from both P. hydropiper (water pepper) and P. maculosa. Good quality RNA was obtained from young flowers from both species and sequenced. The sequence quality read distribution appeared good and initial comparative screening allowed the identification of a sesquiterpene synthase with much higher apparent abundance in P. hydropiper compared to P. maculosa. The putative drimenol synthase (DS) gene was cloned based on the primers 5′-ATGTCTACTGCCGTTAACG-3′ and 5′-CTAAATCGGAATGGGATCGGTG-3′ and expressed in yeast and transiently expressed in Nicotiana benthamiana as described below.

### Example 2

### Expression of Sesquiterpene Synthases in Yeast

The putative full length drimenol synthase gene was cloned into pYES3CT yeast expression vector (Invitrogen) with the TRP1 selection marker using HindIII and NotI restriction sites. The vector was transformed into the into yeast strain WAT11 expressing Arabidopsis ATR1 NADPH-cytochrome P450 reductase. After transformation yeast clones containing the drimenol synthase were selected on Synthetic Dextrose minimal medium (0.67% Difco yeast nitrogen base medium without amino acids, 2% d-glucose, 2% agar) supplemented with amino acids, but omitting L-tryptophane for auxotrophic selection of transformants.

A starter yeast culture was grown overnight at 30° C. in 5 ml of Synthetic Galactose minimal medium (0.67% Difco yeast nitrogen base medium without amino acids, 2% d-galactose, amino acids, but omitting L-tryptophane). The starter culture was diluted to OD600 of 0.05 in 50 ml of Synthetic Galactose minimal medium and incubated at 200 rpm at 30° C. The culture was overlaid with 5 ml of n-dodecane when the OD600 was in the range from 0.8 to 1 and cultivation was continued for 3 days. The n-dodecane layer was collected and centrifuged at 1200 rpm for 10 min, diluted threefold in ethyl acetate, dried using anhydrous Na2SO4 and then used for GC-MS analysis.

### Example 3

### Agrobacterium Transformation

### Construction of Binary Vectors.

Mitochondria targeting: The following genes were cloned into the binary vector pBinplus:
1) the drimenol synthase gene from V. officinalis DSval 2.5 (SEQ ID NO:1) linked to the Cox IV secretion signal from S. cerevisiae for targeting mitochondria (Kohler et al. 1997 Plant J 11: 613; Gene ID:852688; SEQ ID NO:7) under control of the 35S promoter;
2) the drimenol synthase gene from P. hydropiper DSph1.5 (SEQ ID NO:3) linked to the nucleotide sequence of SEQ ID NO:7 for targeting mitochondria under control of the Chrysanthemum rbcS1 promoter (Outchkourov et al. 2003 Planta 216: 1003).
3) the farnesyldiphosphate synthase gene from A. thaliana (Genebank: NM_{—}117823, 1026 bp) FPS1.5 coding for the amino acid sequence of SEQ ID NO:6 linked to the mitochondrial targeting sequence of SEQ ID NO:7 under control of the Chrysanthemum rbcS1 promoter. 4) the 3-hydroxy-3-methylglutaryl-CoA reductase gene from A. thaliana (accession number J04537, 2195bp) HMGR1.1 coding for the truncated amino acid sequence (aa 165-592; SEQ ID NO:5).
Plastids targeting. Alternatively, the following constructs for targeting plastids were cloned into the pBinplus vectors:
1) the drimenol synthase gene from P. hydropiper DSph1.4 (SEQ ID NO:3) linked to the nucleotide sequence coding for the secretion signal from the Chrysantemum mortifolium small subunit protein (Wong et al. 1992 Plant Mol Biol 20: 81-93; SEQ ID NO:8) under control of Rbsc promoter;
2) the farnesyldiphosphate synthase gene from A. thaliana (genebank: NM_{—}117823, 1026 bp) FPS1.4 coding for a nucleic acid of SEQ ID NO:7 linked to the nucleotide sequence of the plastid targeting sequence as set forth in SEQ ID NO:8;
3) hydroxy-3-methylglutaryl-CoA reductase gene from A. thaliana (accession number J04537, 2195bp) HMGR1.1 coding for the truncated amino acid sequence (aa 165-592; SEQ ID NO:6).
Cytosol targeting. Finally, each the following genes DSph1.1. FPS1.1 and HMGR1.1 without sequences coding for organelle targeting peptides were cloned into the pBinplus vector.
   The binary vectors were introduced into the Agl-1 Agrobacterium tumefaciens strain by electroporation. The Agl-1 strain contains a disarmed Ti plasmid that provides the vir gene function and harbors the chromosome markers rifampicin and carbenicillin (Hellens et al. 2000 Trends in Plant Science 5: 446).

### Example 4

### Transient Expression in Leaves of Nicotiana Benthamiana

Agrobacterium strains were grown at 28° C. at 220 rpm for 24 hours in LB media with kanamycin (50 mg/L) and rifampicillin (34 mg/L). Cells were harvested by centrifugation for 20 min at 4,000 g and 20° C. and then resuspended in 10 mM MES buffer containing 10 mM MgCl2 and 100 μM acetosyringone (4′-hydroxy-3′,5′-dimethoxyacetophenone, Sigma) to a final OD600 of 0.5, followed by incubation at room temperature and 50 rpm for 150 minutes. For co-infiltration, equal volumes of the Agrobacterium strains were mixed. Nicotiana benthamiana plants were grown from seeds on soil in a greenhouse with 16 h light at 28° C. (16 h)/25° C. (8 h). Strain mixtures were infiltrated into leaves of four-week-old N. benthamiana plants using a 1 mL syringe. The bacteria were slowly injected into the abaxial side of the leaf. The plants were grown and infiltrated leaves were collected 5 days after infiltration.

Compounds accumulated in the leaves were analyzed by snap freezing and grinding 500 mg infiltrated leaf from plant in liquid nitrogen and extraction with 2 ml dichloromethane. The extracts were prepared by brief vortexing and sonication for 5 min. Then the extracts were centrifuged for 10 min at 1,200 rpm and the clear part of the solution was transferred to a fresh vial. Finally, the extracts were concentrated by evaporating the solvent to a volume of about 0.5 mL and dehydrated using anhydrous Na2SO4. Analysis of the samples was performed by GC-MS (Agilent GC-MS, Agilent technologies). Program was set to 5 min/300° C., 10° C/s temperature increase, 12.5 min solvent delay.

### Example 5

### GC-MS Analysis

Analytes from 1 μL samples were separated using a gas chromatograph (5890 series II, Hewlett-Packard) equipped with a 30 m×0.25 mm, 0.25 mm film thickness column (ZB-5, Phenomenex) using helium as carrier gas at flow rate of 1 ml/min. The injector was used in splitless mode with the inlet temperature set to 250° C. The initial oven temperature of 45° C. was increased after 1 min to 300° C. at a rate of 10° C./min and held for 5 min at 300° C. The GC was coupled to a mass-selective detector (model 5972A, Hewlett-Packard). Compounds were identified by comparison of mass spectra and retention times (rt) with those of the authentic standards comprising for example drimenol.

### Example 6

### In Vivo Production of Drimenol Using DSval and DSph

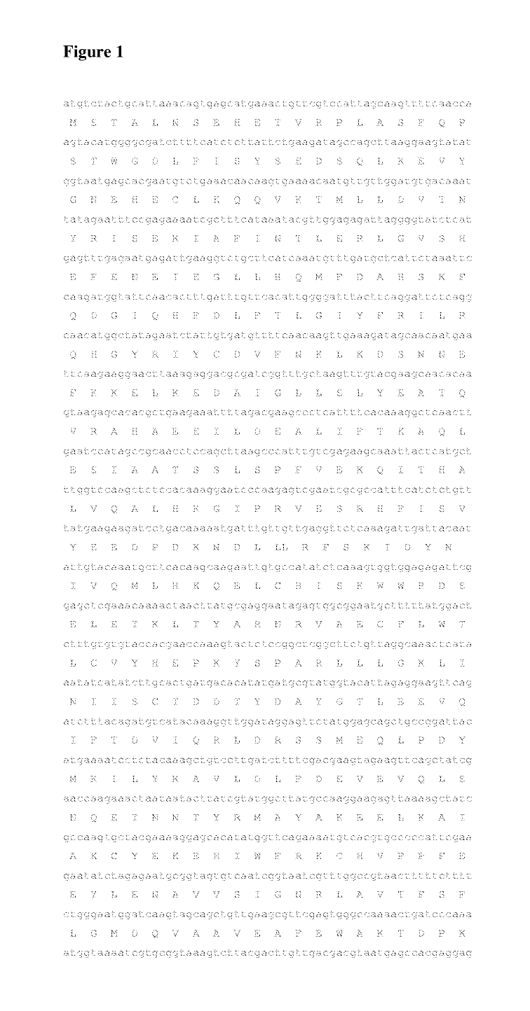

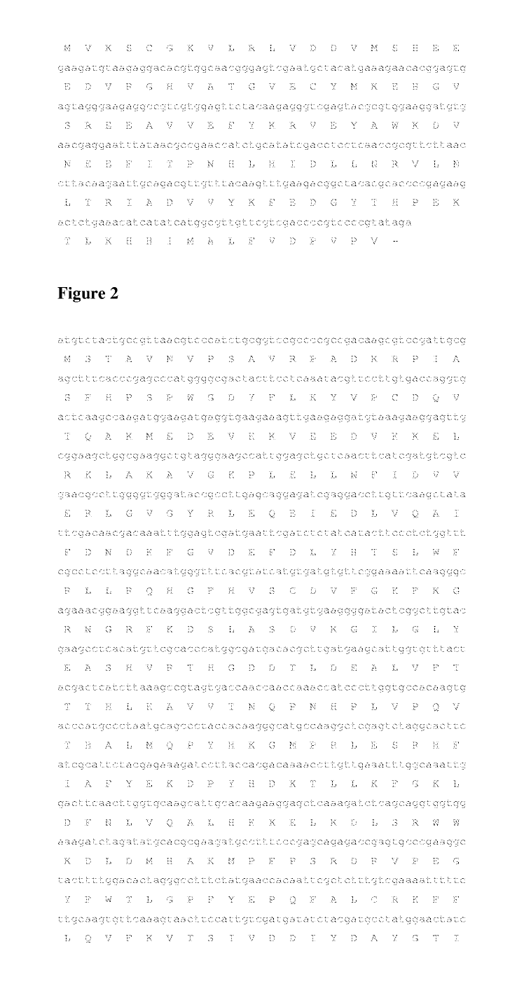

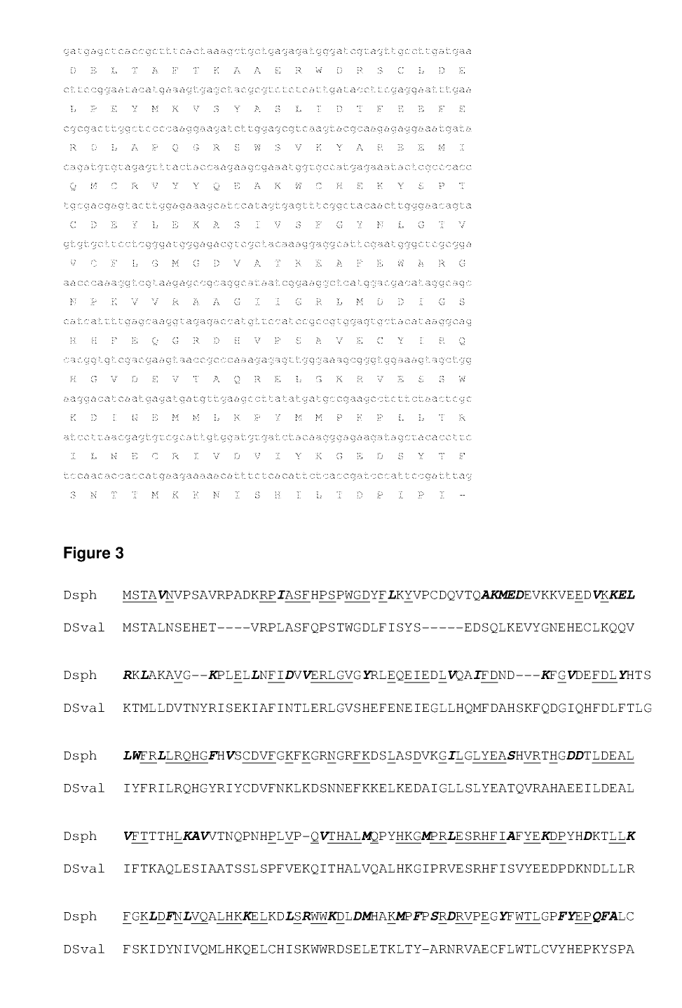

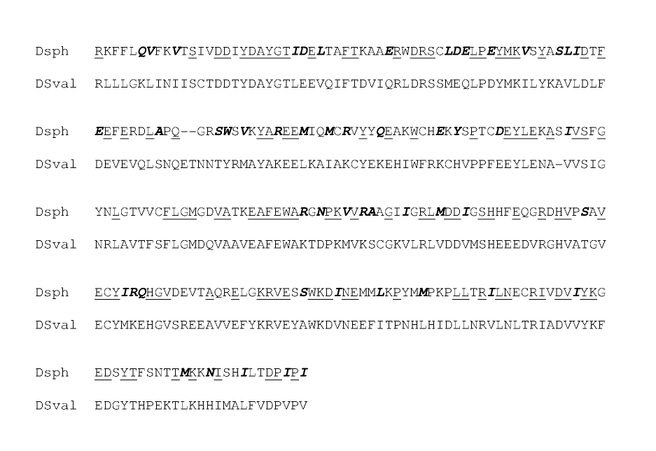

The drimenol synthase (DS) mRNA from V. officinalis (Val) encodes a protein of 556 amino acid residues (FIG. 1) while the DS mRNA from P. hydropiper (Ph) encodes a protein of 559 amino acid residues (FIG. 2). Both genes show little sequence homology (41% identity), see FIG. 3.

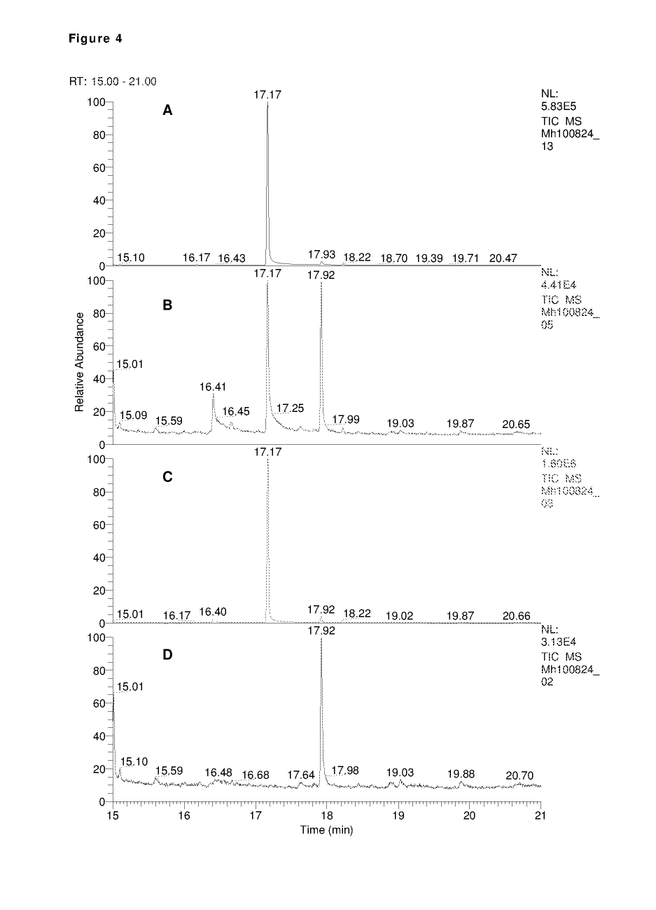

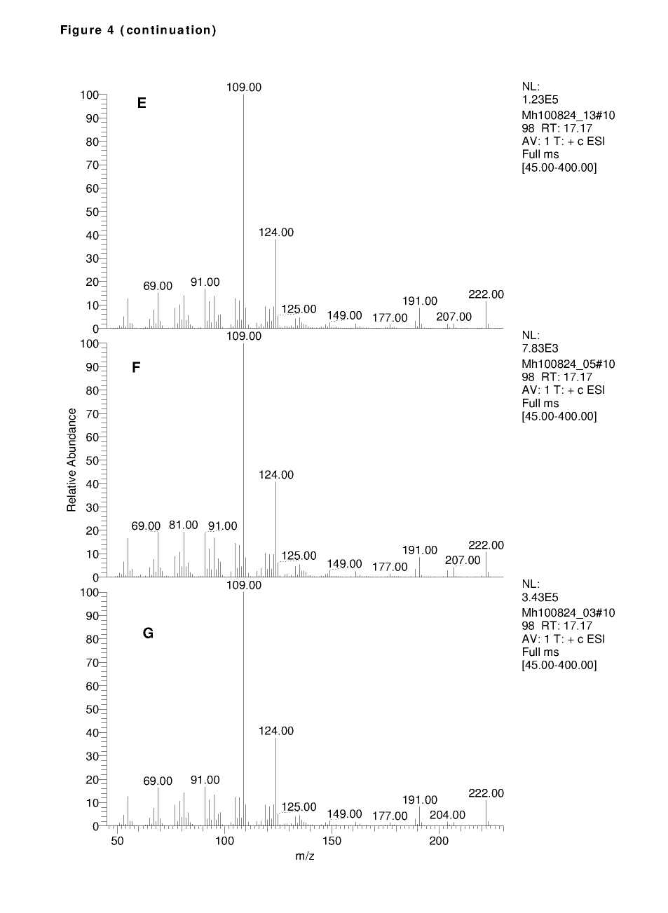

The activity of both drimenol synthases were tested by expression in yeast in yeast transformation experiment and GC-MS analysis as described above. Extracts from cultures of yeast cells transformed with plasmids containing both drimenol synthase genes revealed the presence of the predominant peak identified as drimenol (FIG. 4; rt=17.17 min).

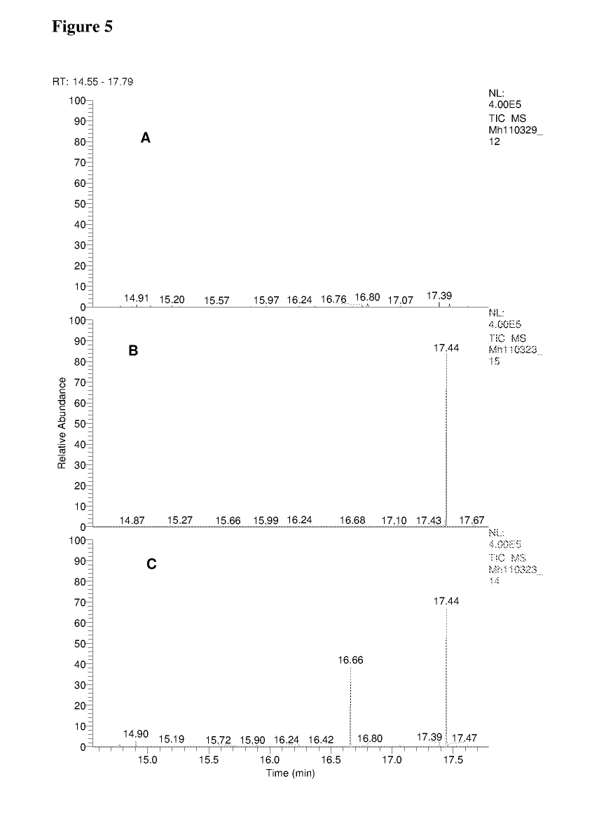

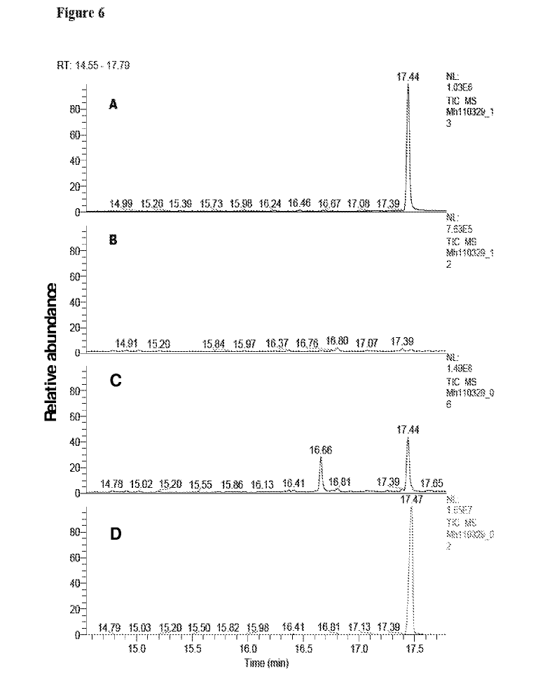

Furthermore, both genes were also transiently expressed in Nicotiana benthamiana leaves. The drimenol synthase from Valeriana officinalis under control of a 35S promoter (FIG. 5) and targeted to the mitochondria, and the drimenol synthase from Persicaria hydropiper under control of Rbcs promoter and targeted to either the plastid or cytosol (FIG. 6). Both DS genes were co-infiltrated with farnesyldiphosphate synthase (FPS) and 3-hydroxy-3-methylglutaryl-CoA reductase (HMGR) which can greatly increase the expression. The results show expression of drimenol in infiltrated leaves by either enzyme (rt=17.44 min). Expression of the native gene in the cytosol without added targeting yielded the most efficient expression of drimenol in N. benthamiana leaves.

### Example 7

### DSval and DSph Sequence Identity

Nucleotide sequence identity was determined using BLASTN, publicly available through the National Center for Biotechnology Information (NCBI) at the web site of the National Institute of Health, USA. BLAST searches were done using the nucleic acid sequences of drimenol synthases from P. hydropiper (1680 bp) and from V. officinalis (1672 bp). The analysis revealed a relatively low sequence identity with a germacrene synthase from poplar (E-value of 0.80). The sequence identity between the P. hydropiper drimenol synthase or V. officinalis drimenol synthase and Vitis vinifera germacrene synthase at the nucleotide level was found to be 52% or 58%, respectively.

Comparison of the translated sequence (BLASTP) of the P. hydropiper drimenol synthase (559 aa) with the NCBI non-redundant database gave an E-value of 2.90E-152 with a germacrene synthase from V. vinifera. The sequence identity between P. hydropiper drimenol synthase and V. vinifera cadenine synthase, valencene synthase and germacrene synthase are 50%, 47% and 42%, respectively. The sequence identity between V. officinalis (556 aa) and V. vinifera germacrene synthase is 51%.

### SEQUENCE LISTING

SEQ ID NO: 1: The nucleotide sequence encoding the drimenol
synthase isolated from V. officinalis
ATGTCTACTGCATTAAACAGTGAGCATGAAACTGTTCGTCCATTAGCAAGTTTTCAACCA
AGTACATGGGGCGATCTTTTCATCTCTTATTCTGAAGATAGCCAGCTTAAGGAAGTATAT
GGTAATGAGCACGAATGTCTGAAACAACAAGTGAAAACAATGTTGTTGGATGTGACAAAT
TATAGAATTTCCGAGAAAATCGCTTTCATAAATACGTTGGAGAGATTAGGGGTATCTCAT
GAGTTTGAGAATGAGATTGAAGGTCTGCTTCATCAAATGTTTGATGCTCATTCTAAATTC
CAAGATGGTATTCAACACTTTGATTTGTTCACATTGGGGATTTACTTCAGGATTCTCAGG
CAACATGGCTATAGAATCTATTGTGATGTTTTCAACAAGTTGAAAGATAGCAACAATGAA
TTCAAGAAGGAACTTAAAGAGGACGCGATCGGTTTGCTAAGTTTGTACGAAGCAACACAA
GTAAGAGCACACGCTGAAGAAATTTTAGACGAAGCCCTCATTTTCACAAAGGCTCAACTT
GAATCCATAGCCGCAACCTCCAGCTTAAGCCCATTTGTCGAGAAGCAAATTACTCATGCT
TTGGTCCAAGCTCTCCACAAAGGAATCCCAAGAGTCGAATCGCGCCATTTCATCTCTGTT
TATGAAGAAGATCCTGACAAAAATGATTTGTTGTTGAGGTTCTCAAAGATTGATTACAAT
ATTGTACAAATGCTTCACAAGCAAGAATTGTGCCATATCTCAAAGTGGTGGAGAGATTCG
GAGCTCGAAACAAAACTAACTTATGCGAGGAATAGAGTGGCGGAATGCTTTTTATGGACT
CTTTGTGTGTACCACGAACCAAAGTACTCTCCGGCTCGGCTTCTGTTAGGCAAACTCATA
AATATCATATCTTGCACTGATGACACATATGATGCGTATGGTACATTAGAGGAAGTTCAG
ATCTTTACAGATGTCATACAAAGGTTGGATAGGAGTTCTATGGAGCAGCTGCCGGATTAC
ATGAAAATCCTCTACAAAGCTGTCCTTGATCTTTTCGACGAAGTAGAAGTTCAGCTATCG
AACCAAGAAACTAATAATACTTATCGTATGGCTTATGCCAAGGAAGAGTTAAAAGCTATC
GCCAAGTGCTACGAAAAGGAGCACATATGGTTCAGAAAATGTCACGTGCCCCCATTCGAA
GAATATCTAGAGAATGCGGTAGTGTCAATCGGTAATCGTTTGGCCGTAACTTTTTCTTTT
CTGGGAATGGATCAAGTAGCAGCTGTTGAAGCGTTCGAGTGGGCCAAAACTGATCCCAAA
ATGGTAAAATCGTGCGGTAAAGTCTTACGACTTGTTGACGACGTAATGAGCCACGAGGAG
GAAGATGTAAGAGGACACGTGGCAACGGGAGTCGAATGCTACATGAAAGAACACGGAGTG
AGTAGGGAAGAGGCCGTCGTGGAGTTCTACAAGAGGGTCGAGTACGCGTGGAAGGATGTG
AACGAGGAATTTATAACGCCGAACCATCTGCATATCGACCTCCTCAACCGCGTTCTTAAC
CTTACAAGAATTGCAGACGTTGTTTACAAGTTTGAAGACGGCTACACGCACCCCGAGAAG
ACTCTGAAACATCATATCATGGCGTTGTTCGTCGACCCCGTCCCCGTATAGA
SEQ ID NO: 2: The amino acid sequence of drimenol synthase
isolated from V. officinalis
MSTALNSEHETVRPLASFQPSTWGDLFISYSEDSQLKEVYGNEHECLKQQVKTMLLDVTN
YRISEKIAFINTLERLGVSHEFENEIEGLLHQMFDAHSKFQDGIQHFDLFTLGIYFRILR
QHGYRIYCDVFNKLKDSNNEFKKELKEDAIGLLSLYEATQVRAHAEEILDEALIFTKAQL
ESIAATSSLSPFVEKQITHALVQALHKGIPRVESRHFISVYEEDPDKNDLLLRFSKIDYN
IVQMLHKQELCHISKWWRDSELETKLTYARNRVAECFLWTLCVYHEPKYSPARLLLGKLI
NIISCTDDTYDAYGTLEEVQIFTDVIQRLDRSSMEQLPDYMKILYKAVLDLFDEVEVQLS
NQETNNTYRMAYAKEELKAIAKCYEKEHIWFRKCHVPPFEEYLENAVVSIGNRLAVTFSF
LGMDQVAAVEAFEWAKTDPKMVKSCGKVLRLVDDVMSHEEEDVRGHVATGVECYMKEHGV
SREEAVVEFYKRVEYAWKDVNEEFITPNHLHDLLNRVLNLTRIADVVYKFEDGYTHPEKT
LKHHIMALFVDPVPV
SEQ ID NO: 3: The nucleotide sequence encoding the drimenol
synthase isolated from P. hydropiper
ATGTCTACTGCCGTTAACGTCCCATCTGCGGTCCGCCCCGCCGACAAGCGTCCGATTGC
GAGCTTTCACCCGAGCCCATGGGGCGACTACTTCCTCAAATACGTTCCTTGTGACCAGG
TGACTCAAGCCAAGATGGAAGATGAGGTGAAGAAAGTTGAAGAGGATGTAAAGAAGGAG
TTGCGGAAGCTGGCGAAGGCTGTAGGGAAGCCATTGGAGCTGCTCAACTTCATCGATGT
CGTCGAACGCCTTGGGGTGGGATACCGCCTTGAGCAGGAGATCGAGGACCTTGTTCAAG
CTATATTCGACAACGACAAATTTGGAGTCGATGAATTCGATCTCTATCATACTTCCCTC
TGGTTTCGCCTCCTTAGGCAACATGGGTTTCACGTATCATGTGATGTGTTCGGAAAATT
CAAGGGCAGAAACGGAAGGTTCAAGGACTCGTTGGCGAGTGATGTGAAGGGGATACTCG
GCTTGTACGAAGCCTCACATGTTCGCACCCATGGCGATGACACGCTTGATGAAGCATTG
GTGTTTACTACGACTCATCTTAAAGCCGTAGTGACCAACCAACCAAACCATCCCTTGGT
GCCACAAGTGACCCATGCCCTAATGCAGCCCTACCACAAGGGCATGCCAAGGCTCGAGT
CTAGGCACTTCATCGCATTCTACGAGAAAGATCCTTACCACGACAAAACCTTGTTGAAA
TTTGGCAAATTGGACTTCAACTTGGTGCAAGCATTGCACAAGAAGGAGCTCAAAGATCT
CAGCAGGTGGTGGAAAGATCTAGATATGCACGCGAAGATGCCTTTCCCGAGCAGAGACC
GAGTGCCCGAAGGCTACTTTTGGACACTAGGGCCTTTCTATGAACCACAATTCGCTCTT
TGTCGAAAATTTTTCTTGCAAGTGTTCAAAGTAACTTCCATTGTCGATGATATCTACGA
TGCCTATGGAACTATCGATGAGCTCACCGCTTTCACTAAAGCTGCTGAGAGATGGGATC
GTAGTTGCCTTGATGAACTTCCGGAATACATGAAAGTGAGCTACGCGTCTCTCATTGAT
ACCTTCGAGGAATTTGAACGCGACTTGGCTCCCCAAGGAAGATCTTGGAGCGTCAAGTA
CGCAAGAGAGGAAATGATACAGATGTGTAGAGTTTACTACCAAGAAGCGAAATGGTGCC
ATGAGAAATACTCGCCCACCTGCGACGAGTACTTGGAGAAAGCATCCATAGTGAGTTTC
GGCTACAACTTGGGAACAGTAGTGTGCTTCCTCGGGATGGGAGACGTCGCTACAAAGGA
GGCATTCGAATGGGCTCGCGGAAACCCAAAGGTCGTAAGAGCCGCAGGCATAATCGGAA
GGCTCATGGACGACATAGGCAGCCATCATTTTGAGCAAGGTAGAGACCATGTTCCATCC
GCCGTGGAGTGCTACATAAGGCAGCACGGTGTCGACGAAGTAACCGCCCAAAGAGAGTT
GGGAAAGCGGGTGGAAAGTAGCTGGAAGGACATCAATGAGATGATGTTGAAGCCTTATA
TGATGCCGAAGCCTCTTCTAACTCGCATCCTTAACGAGTGTCGCATTGTGGATGTGATC
TACAAGGGAGAAGATAGCTACACCTTCTCCAACACCACCATGAAGAAAAACATTTCTCA
CATTCTCACCGATCCCATTCCGATTTAG
SEQ ID NO: 4: The amino acid sequence of drimenol synthase
isolated from P. hydropiper
MSTAVNVPSAVRPADKRPIASFHPSPWGDYFLKYVPCDQVTQAKMEDEVKKVEEDVKKEL
RKLAKAVGKPLELLNFIDVVERLGVGYRLEQEIEDLVQAIFDNDKFGVDEFDLYHTSLWF
RLLRQHGFHVSCDVFGKFKGRNGRFKDSLASDVKGILGLYEASHVRTHGDDTLDEALVFT
TTHLKAVVINQPNHPLVPQVTHALMQPYHKGMPRLESRHFIAFYEKDPYHDKTLLKFGKL
DFNLVQALHKKELKDLSRWWKDLDMHAKMPFPSRDRVPEGFWTLGPFYEPQFALCRKFFL
QVFKVTSIVDDIYDAYGTIDELTAFTKAAERWDRSCLDELPEYMKVSYASLIDTFEEFER
DLAPQGRSWSVKYAREEMIQMCRVYYQEAKWCHEKYSPTCDEYLEKASIVSFGYNLGTVV
CFLGMGDVATKEAFEWARGNPKVVRAAGIIGRLMDDIGSHHFEQGRDHVPSAVECYIRQH
GVDEVTAQRELGKRVESSWKDINEMMLKPYMMPKPLLTRILNECRIVDVIYKGEDSYTFS
NTTMKKNISHILTDPIPI
SEQ ID NO: 5: The amino acid sequence of HMGR isolated
from A. thaliana
MDPTESLPEEDEEIVKSVIDGVIPSYSLESRLGDCKRAASIRREALQRVTGRSIEGLPLD
GFDYESILGQCCEMPVGYIQIPVGIAGPLLLDGYEYSVPMATTEGCLVASTNRGCKAMFI
SGGATSTVLKDGMTRAPVVRFASARRASELKFFLENPENFDTLAVVFNRSSRFARLQSVK
CTIAGKNAYVRFCCSTGDAMGMNMVSKGVQNVLEYLTDDFPDMDVIGISGNFCSDKKPAA
VNWIEGRGKSVVCEAVIRGEIVNKVLKTSVAALVELNMLKNLAGSAVAGSLGGFNAHASN
IVSAVFIATGQDPAQNVESSQCITMMEAINDGKDIHISVTMPSIEVGTVGGGTQLASQSA
CLNLLGVKGASTESPGMNARRLATIVAGAVLAGELSLMSAIAAGQLVRSHMKYNRSSRDI
SGATTTTTTTTAAADLQ
SEQ ID NO: 6: The amino acid sequence of FPS2 isolated
from A. thaliana
MADLKSTFLDVYSVLKSDLLQDPSFEFTHESRQWLERMLDYNVRGGKLNRGLSWDSYKLL
KQGQDLTEKETFLSCALGWCIEWLQAYFLVLDDIMDNSVTRRGQPCWFRKPKVGMIAIND
GILLRNHIHRILKKHFREMPYYVDLVDLFNEVEFQTACGQMIDLITTFDGEKDLSKYSLQ
IHRRIVEYKTAYYSFYLPVACALLMAGENLENHTDVKTVLVDMGIYFQVQDDYLDCFADP
ETLGKIGTDIEDFKCSWLVVKALERCSEEQTKILYENYGKAEPSNVAKVKALYKELDLEG
AFMEYEKESYEKLTKLIEAHQSKAIQAVLKSFLAKIYKRQK
SEQ ID NO: 7: The nucleotide sequence encoding
Cox IV secretion signal isolated from
S. cerevisiae (mitochondrial target sequence)
ATGTTGTCACTACGTCAATCTATAAGATTTTTCAAGCCAGCCACAAGAACTTTGTG
TAGCTCTCGTTATCTG
SEQ ID NO: 8: The nucleotide sequence encoding the
secretion signal isolated from the Ch. morifolium
small subunit protein
ATGGCCTCGATCTCTTCCTCCGCTGTCGCAACCGTCAACAGGACCACCTCTGCTCAAGC
TAGCATGGTGGCTCCATTCACCGGGCTTAAGTCCAACGTCGCTTTCCCAGTCACCAAGA
AGTCTAACGACTTCTCATCCCTCCCCAGCAACGGTGGAAGAGTGCAATGCATGAAGGTA
CAATATATAACTTAAATAATAACGTGAACACTTATTATAATGCAGTAGATATAATGACT
AACATTTTATAAAATATATATATAGGTGTGGCCACCATTGGGTTTGAAGAAGT

1-23. (canceled)

24. An isolated polypeptide having drimenol synthase activity and comprising the amino acid sequence of SEQ ID NO:2, an amino acid sequence having at least 70% identity with the amino acid sequence of SEQ ID NO:2, the amino acid sequence of SEQ ID NO:4, or an amino acid sequence having at least 70% identity with the amino acid sequence of SEQ ID NO:4.

25. An isolated nucleic acid sequence encoding the polypeptide according to claim 24.

26. The isolated nucleic acid sequence according to claim 25 comprising a nucleotide sequence of SEQ ID NO:1 or SEQ ID NO:3.

27. The isolated nucleic acid sequence according to claim 25 derived from Valeriana officinalis or Persicaria hydropiper.

28. A chimeric gene comprising the nucleic acid sequence according to claim 25.

29. An expression vector comprising a nucleic acid sequence according to claim 25.

30. The expression vector according to claim 29, operably linked to at least one regulatory sequence which controls transcription, translation initiation or termination.

31. The expression vector according to claim 29, wherein the nucleic acid sequence further comprises a targeting sequence.

32. The expression vector according to claim 31, wherein the targeting sequence encodes a transit peptide that targets a polypeptide having drimenol synthase activity and comprising the amino acid sequence of SEQ ID NO:2, an amino acid sequence having at least 70% identity with the amino acid sequence of SEQ ID NO:2, the amino acid sequence of SEQ ID NO:4, or an amino acid sequence having at least 70% identity with the amino acid sequence of SEQ ID NO:4, to a plastid or a mitochondria of a plant cell.

33. The expression vector according to claim 32, wherein the plastid is a chloroplast.

34. A host cell comprising an isolated nucleic acid sequence according to claim 25.

35. The host cell according to claim 34, wherein the cell is a prokaryotic or eukaryotic cell, such as a mammalian cell, a bacterial cell, a fungal cell, or a plant cell.

36. A transgenic organism comprising an isolated nucleic acid sequence according to claim 25.

37. The organism according to claim 34, wherein the organism is a plant.

38. A method for producing drimenol, and/or at least one drimenol derivative, comprising:
a) contacting a farnesyl diphosphate (FPP) precursor with a polypeptide according to claim 24 under conditions allowing conversion of FPP to drimenol;
b) isolating drimenol; and
c) optionally, contacting drimenol produced in steps (a), (b) with at least one enzyme to convert drimenol to the at least one drimenol derivative.

39. The method according to claim 38, further comprising, prior to step (a): transfecting and/or transforming a cell capable of producing the FPP precursor with a nucleic acid sequence encoding a polypeptide having drimenol synthase activity and comprising the amino acid sequence of SEQ ID NO:2, an amino acid sequence having at least 70% identity with the amino acid sequence of SEQ ID NO:2, the amino acid sequence of SEQ ID NO:4, or an amino acid sequence having at least 70% identity with the amino acid sequence of SEQ ID NO:4, to provide for a cell capable of producing drimenol.

40. The method according to claim 38, wherein step (a) is carried out by culturing the cell under conditions allowing production of drimenol and/or the drimenol derivative.

41. The method according to claim 38, wherein the cell is selected from the group consisting of a plant cell, bacterial cell or fungal cell.

42. The method according to claim 38, further comprising hydroxylating and/or oxidizing of drimenol to produce at least one drimenol derivative having fungicidal, insecticidal, antifeedant, fragrance and or food taste modifying properties.

43. The method according to claim 38, wherein the drimenol derivative is selected from the group consisting of driman-8-ol, driman-8,11-diol, drim-8-en-7-one, forskolin, cinnamodial, (+)-albicanol, (−)-uvidin, (+)-isopolygonal, (−)-polygodial, (−)-ugandensidial, (−)-warburganal, ambergris, drimenal, drimenoic acid, isodrimenin, cinnamolide, confertolin, confertifolin, drimendiol, and polygodial acid.

44. A method for producing a polypeptide having drimenol synthase activity, comprising:
a) transforming or transfecting a host cell or a non-human organism with a nucleic acid according to claim 25;
b) culturing the host cell or the organism under conditions allowing production of the polypeptide.

45. A method for producing a transgenic plant capable of producing drimenol, comprising:
a) transforming or transfecting a plant or a plant cell with a nucleic acid according to claim 25;
b) regenerating a transgenic plant from the transformed or transfected plant or plant cell.

46. The method according to claim 45, further comprising:
c) screening the transgenic plant, or a plant derived therefrom by selfing or crossing, for production of drimenol and identifying a transgenic plant producing drimenol.

47. A crop plant comprising the nucleic acid sequence according to claim 25.

48. A crop plant according to claim 47, which is selected from the group consisting of tomato, pepper, eggplant, lettuce, sunflower, oilseed rape, broccoli, cauliflower and cabbage crops, cucumber, melon, watermelon, pumpkin, squash, peanut, soybeans, cotton, beans, cassava, potatoes, sweet potato, okra, maize, barley, pearl millet, wheat, rye, sorghum, and rice.
